(19) United States
(12) Patent Application Publication
(10) Pub. No.: US 2005/0277663 A1
(43) Pub. Date: Dec. 15, 2005

(54) 3-Aryl-3-hydroxy-and 3-aryl-3-oxopropionic acid esters as fungicides

(76) Inventors: Shy-Fuh Lee, Sunnyvale, CA (US); Takeo Hokama, Mountain View, CA (US); Richard Anderson, Palo Alto, CA (US)

Correspondence Address: MYERS BIGEL SIBLEY & SAJOVEC, PO BOX 37428, RALEIGH, NC, 27627, US

(21) Appl. No.: 11/152,304

(22) Filed: Jun. 14, 2005

Related U.S. Application Data
(60) Provisional application No. 60/579,820, filed on Jun. 15, 2004.

(51) Int. Cl. A61K 31/4745; A61K 31/444; A61K 31/4439; C07D 471/02; C07D 41/02

(52) U.S. Cl. 514/303; 514/332; 514/340; 546/118; 546/269.7; 546/255

(57) ABSTRACT

Compounds of formula I: 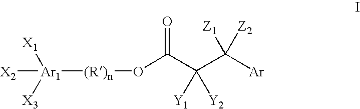
wherein Ar is aryl, Ar₁ is a 5 or 6-membered aromatic or heteroaromatic group, Z₁ is —OH and Z₂ is H; or Z₁ and Z₂ together are ═O; are described, along with methods of making the same, compositions containing the same, and methods of using the same, particularly as fungicides.

### RELATED APPLICATIONS

This application claims the benefit of U.S. provisional patent application Ser. No. 60/579,820, filed Jun. 15, 2004, the disclosure of which is incorporated by reference herein in its entirety.

### FIELD OF THE INVENTION

The present invention concerns substituted 3-hydroxy- and 3-oxopropionic acid esters, compositions thereof, and methods of use thereof for the control of microbial pests, particularly fungal pests, on plants.

### BACKGROUND OF THE INVENTION

The incidence of serious fungal infections, either systemic or topical, continues to increase for plants, animals, and humans. Many fungi are common in the environment and not harmful to plants or mammals. However, some fungi can produce disease in plants, humans and/or animals.

Fungicides are compounds, of natural or synthetic origin, which act to protect plants against damage caused by fungi, including oomycetes. Current methods of agriculture rely heavily on the use of fungicides. In fact, some crops cannot be grown usefully without the use of fungicides. Using fungicides allows a grower to increase the yield of the crop and consequently, increase the value of the crop. Numerous fungicidal agents have been developed. However, the treatment of fungal infestations and infections continues to be a major problem. Furthermore, fungicide and antifungal drug resistance has become a serious problem, rendering these agents ineffective for some agricultural and therapeutic uses. As such, a need exists for the development of new fungicidal and antifungal compounds (see, e.g., U.S. Pat. No. 6,673,827; See also U.S. Pat. No. 6,617,330 to Walter, which describes pyrimidin-4-enamine as fungicides).

### SUMMARY OF THE INVENTION

The present invention relates to the field of fungicidal compositions and methods. More particularly, the present invention concerns novel fungicidal 3-hydroxy- and 3-oxopropionic acid esters and methods involving application and use of fungicidally effective amounts of such compounds. The present invention also concerns methods useful in the preparation of such 3-hydroxy- and 3-oxopropionic acid esters.

The compounds and compositions of the present invention are useful as crop protection agents to combat or prevent fungal infestations, or to control other pests such as weeds, insects, or acarids that are harmful to crops.

Accordingly, a first aspect of the present invention is a compound of formula I: 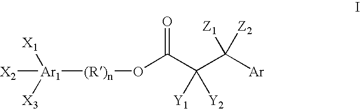
wherein:
Z₁ is —OH and Z₂ is H; or Z₁ and Z₂ together are ═O;
Y₁ and Y₂ are each independently selected from the group consisting of H, alkyl, alkynyl, haloalkyl, alkoxyalkyl, alkylthioalkyl, alkoxy, aryloxy, cyano, R₂R₃NCH₂—, arylthioalkyl, N-heterocycloalkyl, aryl and arylalkyl, each of which can be optionally substituted with halogen, alkyl, alkynyl, haloalkyl, alkoxy, alkylthio, haloalkoxy, cyano, nitro, alkylsulfinyl, or alkylsulfonyl;
or Y₁ and Y₂ taken together form a 3- to 6-membered ring comprised of 3-6 C-atoms, 1-3 N-atoms, and 0-1 O-atom, optionally substituted by alkyl, alkoxycarbonyl, aryl;
Ar is aryl;
Ar₁ is a 5 or 6-membered aromatic or heteroaromatic ring;
X₁, X₂, and X₃ are each taken individually from the group consisting of H, halogen, alkyl, alkenyl, alkynyl, haloalkyl, alkoxy, haloalkoxy, alkylthio, cyano, nitro; aryl or aryloxy or aryloxyalkyl, optionally substituted with halogen, alkyl, haloalkyl, alkoxy, alkylthio, haloalkoxy, cyano, and nitro;
or X₁ and X₂ or X₂ and X₃ as an adjacent pair together form the group —Y—W-Z-, where Y and Z are independently oxygen, sulphur, sulphonyl, carbonyl, or CR₄R₅, W is —(CR₆R₇)ₚ—(CR₈R₉)_{q}— or sulfonyl, R₄ and R₅ are independently hydrogen, halogen, or alkyl, R₆ and R₇ are independently hydrogen, halogen, alkyl or haloalkyl or together form an oxo group, R₈ and R₉ are independently hydrogen, halogen, alkyl or haloalkyl, p is 1 or 2, and q is 0 or 1;
R′ is C1-C4 alkylene optionally substituted with alkyl, haloalkyl, alkoxy, or cyano;
R₂ and R₃ are each taken individually from alkyl, haloalkyl, aryl optionally substituted with halogen, alkyl, alkynyl, haloalkyl, alkoxy, alkylthio, haloalkoxy, cyano, nitro;
or R₂ and R₃ taken together to form a 5 or 6-member heterocycle containing 2-5 C-atoms, 0-1 O-atom, 0-1 S-atom, and 1-3 N-atoms;
n is 0 or 1;
or a salt thereof.

In some embodiments of the foregoing, Z₁ is —OH and Z₂ is H (see Formula Ia below); in other embodiments of the foregoing, Z₁ and Z₂ together are ═O (see Formula II below).

In some embodiments of the foregoing, Y₁ and Y₂ are H or alkyl; in other embodiments of the foregoing, at least one of Y₁ and Y₂ are arylalkyl or arylthioalkyl. In still other embodiments, at least one of Y₁ and Y₂ is aryl (including substituted aryl).

In some embodiments of the foregoing, Ar is phenyl, optionally substituted with halogen, alkyl, alkynyl, haloalkyl, alkoxy, alkylthio, haloalkoxy, cyano or nitro; in other embodiments of the foregoing, Ar is pyridyl optionally substituted with halogen, alkyl, alkynyl, haloalkyl, alkoxy, alkylthio, haloalkoxy, cyano or nitro; in still other embodiments of the foregoing, Ar is thiazolyl optionally substituted with halogen, alkyl, alkynyl, haloalkyl, alkoxy, alkylthio, haloalkoxy, cyano or nitro.

In some embodiments of the foregoing, Ar₁ is selected from the group consisting of phenyl, pyridyl, thienyl, and furyl.

A second aspect of the present invention is a composition for controlling and preventing plant pathogenic microorganisms comprising, in combination, air active compound as described herein together with a suitable carrier.

A third aspect of the present invention is a method of controlling or preventing infestation of cultivated plants by pathogenic microorganisms, comprising applying an active compound as described herein to said plants, parts thereof or the locus thereof in an amount effective to control said microorganisms.

A further aspect of the present invention is a method of controlling or preventing infestation of plant propagation material by pathogenic microorganisms, comprising: applying an active compound as described herein to said plant propagation material in an amount effective to control said microorganisms.

A further aspect of the present invention is a method of controlling or preventing infestation of plant propagation material or cultivated plants by pathogenic microorganisms comprising applying an active compound as described herein to said plant propagation material or plants, parts thereof or the locus thereof in combination with one or more other fungicides in amounts effective to control said microorganisms.

A further aspect of the present invention is a method of controlling or preventing infestation of technical materials by pathogenic microorganisms, comprising applying an active compound as described herein to said technical materials, parts thereof or the locus thereof in an amount effective to control said microorganisms.

A further aspect of the present invention is a method of treating a fungal infection in a subject in need thereof, comprising administering an active compound as described herein to said subject in an amount effective to treat said fungal infection.

A still further aspect of the present invention is the use of an active compound as described herein for the preparation of a composition (e.g., an agricultural formulation, a pharmaceutical formulation) for carrying out a method as described herein (e.g., an agricultural treatment as described herein, the treatment of technical materials as described herein, the treatment of a fungal infection in a subject as described herein).

The foregoing and other objects and aspects of the present invention are explained in greater detail below.

### DETAILED DESCRIPTION OF THE PREFERRED EMBODIMENTS

“Alkyl” as used herein refers to a saturated hydrocarbon radical which may be straight-chain or branched-chain (for example, ethyl, isopropyl, t-amyl, or 2,5-dimethylhexyl) or cyclic (for example cyclobutyl, cyclopropyl or cyclopentyl) and contains from 1 to 24 carbon atoms. This definition applies both when the term is used alone and when it is used as part of a compound term, such as “haloalkyl” and similar terms. In some embodiments, preferred alkyl groups are those containing 1 to 4 carbon atoms, which are also referred to as “lower alkyl.” In some embodiments preferred alkyl groups are those containing 5 or 6 to 24 carbon atoms, which may also be referred to as “higher alkyl”.

“Alkenyl,” as used herein, refers to a straight or branched chain hydrocarbon containing from 2 to 24 carbons and containing at least one carbon-carbon double bond formed by the removal of two hydrogens. Representative examples of “alkenyl” include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl and the like. “Lower alkenyl” as used herein, is a subset of alkenyl and refers to a straight or branched chain hydrocarbon group containing from 1 to 4 carbon atoms.

“Alkynyl,” as used herein, refers to a straight or branched chain hydrocarbon group containing from 2 to 24 carbon atoms and containing at least one carbon-carbon triple bond. Representative examples of alkynyl include, but are not limited, to acetylenyl, 1-propynyl, 2-propynyl, 3-butynyl, 2-pentynyl, 1-butynyl and the like. “Lower alkynyl” as used herein, is a subset of alkyl and refers to a straight or branched chain hydrocarbon group containing from 1 to 4 carbon atoms.

“Alkoxy” refers to an alkyl radical as described above which also bears an oxygen substituent which is capable of covalent attachment to another hydrocarbon radical (such as, for example, methoxy, ethoxy and t-butoxy).

“Alkylthio” as used herein refers to an alkyl group, as defined herein, appended to the parent molecular moiety through a thio moiety, as defined herein. Representative examples of alkylthio include, but are not limited, methylthio, ethylthio, tert-butylthio, hexylthio, and the like.

“Aryl” or “aromatic ring moiety” refers to an aromatic substituent which may be a single ring or multiple rings which are fused together, linked covalently or linked to a common group such as an ethylene or methylene moiety. The aromatic rings may each contain heteroatoms and hence “aryl” encompasses “heteroaryl” as used herein. Representative examples of aryl include, azulenyl, indanyl, indenyl, naphthyl, phenyl, tetrahydronaphthyl, biphenyl, diphenylmethyl, 2,2-diphenyl-1-ethyl, thienyl, pyridyl and quinoxalyl. “Aryl” means substituted or unsubstituted aryl unless otherwise indicated and hence the aryl moieties may be optionally substituted with halogen atoms, or other groups such as nitro, carboxyl, alkoxy, phenoxy and the like. Additionally, the aryl radicals may be attached to other moieties at any position on the aryl radical which would otherwise be occupied by a hydrogen atom (such as, for example, 2-pyridyl, 3-pyridyl and 4-pyridyl).

“Heteroaryl” means a cyclic, aromatic hydrocarbon in which one or more carbon atoms have been replaced with heteroatoms. If the heteroaryl group contains more than one heteroatom, the heteroatoms may be the same or different. Examples of heteroaryl groups include pyridyl, pyrimidinyl, imidazolyl, thienyl, furyl, pyrazinyl, pyrrolyl, pyranyl, isobenzofuranyl, chromenyl, xanthenyl, indolyl, isoindolyl, indolizinyl, triazolyl, pyridazinyl, indazolyl, purinyl, quinolizinyl, isoquinolyl, quinolyl, phthalazinyl, naphthyridinyl, quinoxalinyl, isothiazolyl, and benzo[b]thienyl. Preferred heteroaryl groups are five and six membered rings and contain from one to three heteroatoms independently selected from O, N, and S. The heteroaryl group, including each heteroatom, can be unsubstituted or substituted with from 1 to 4 substituents, as chemically feasible. For example, the heteroatom S may be substituted with one or two oxo groups, which may be shown as ═O.

“Agriculturally acceptable salt” means a salt the cation of which is known and accepted in the art for the formation of salts for agricultural or horticultural use. Preferably the salts are water-soluble.

“Cyano” as used herein refers to a —CN group.

“Halo” or “halogen,” as used herein, refers to —Cl, —Br, —I or —F.

“Haloalkyl,” as used herein, refers to at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, 2-chloro-3-fluoropentyl, and the like.

“Hydroxy,” as used herein, refers to an —OH group.

“Nitro,” as used herein, refers to a —NO₂ group.

“Oxy,” as used herein, refers to a —O— moiety.

“Thio,” as used herein, refers to a —S— moiety.

The disclosures of all U.S. patent references cited herein are to be incorporated herein in their entirety as if fully set forth.

2. Compounds. The compounds of this invention are represented by formulas I, Ia and II: 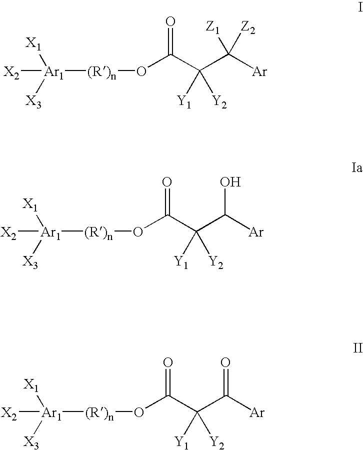
wherein:
Z₁ is —OH and Z₂ is H (as in Formula Ia); or Z₁ and Z₂ together are ═O (as in Formula II);
Y₁ and Y₂ are taken individually from H, alkyl, alkynyl, haloalkyl, alkoxyalkyl, alkylthioalkyl, (substituted)aminoalkyl, alkoxy, aryloxy, or cyano; arylthioalkyl (this term including heteroarylthioalkyl), N-heterocycloalkyl, aryl (this term including heteroaryl) or arylalkyl (this term including heteroarylalkyl) optionally substituted with halogen, alkyl, alkynyl, haloalkyl, alkoxy, alkylthio, haloalkoxy, cyano, nitro, alkylsulfinyl, or alkylsulfonyl;
or Y₁ and Y₂ taken together may be part of a 3- to 6-membered ring comprised of 3-6 C-atoms, 1-3 N-atoms, and 0-1 O-atom, optionally substituted by alkyl, alkoxycarbonyl, or aryl (heteroaryl).

Ar means aryl, preferably phenyl optionally substituted with halogen, alkyl, alkynyl, haloalkyl, alkoxy, alkylthio, haloalkoxy, cyano, nitro; heteroaryl, especially 2-, 3- or 4-pyridyl optionally substituted with halogen, alkyl, alkynyl, haloalkyl, alkoxy, alkylthio, haloalkoxy, cyano, nitro; 2-thiazolyl, 5-thiazolyl, or 5-(1,2,3-thiadiazolyl) optionally substituted with halogen, alkyl, alkynyl, alkoxy, alkylthio, haloalkyl, cyano, nitro;
Ar₁ means aryl, preferably a 5- or 6-membered aromatic ring comprised of 2-6 C-atoms, 0-3 N-atoms, 0-1 O-atom, and 0-1 S-atom, especially phenyl, pyridyl, thienyl, or furyl;
X₁, X₂, and X₃ are taken individually from H, halogen, alkyl, alkenyl, alkynyl, haloalkyl, alkoxy, haloalkoxy, alkylthio, cyano, nitro; aryl or aryloxy or aryloxyalkyl, optionally substituted with halogen, alkyl, haloalkyl, alkoxy, alkylthio, haloalkoxy, cyano, or nitro,
or X₁ and X₂ or X₂ and X₃ as an adjacent pair together form the group —Y—W-Z-, where Y and Z are independently oxygen, sulphur, sulphonyl, carbonyl, or CR₄R₅, W is —(CR₆R₇)ₚ—(CR₈R₉)_{q}— or sulfonyl, R₄ and R₅ are independently hydrogen, halogen, or alkyl, R₆ and R₇ are independently hydrogen, halogen, alkyl or haloalkyl or together form an oxo group, R₈ and R₉ are independently hydrogen, halogen, alkyl or haloalkyl, p is 1 or 2, and q is 0 or 1;
R′ is C1-C4 alkylene optionally substituted with alkyl, haloalkyl, alkoxy, or cyano;
n is 0 or 1.

Methods of making. Compositions of formula Ia where Y₁ and/or Y₂=alkyl or arylalkyl may be prepared by the aldol condensation of an ester of formula III with an aldehyde of formula IV. The requisite ester enolate for this aldol condensation may be formed using a strong base such as lithium diisopropylamide at low temperature (typically −40° C. to −78° C.) in an ether solvent such as tetrahydrofuran. Addition of the aldehyde IV to the enolate is done at low temperature. Compositions I in which Y₁=alkyl and Y₂=H, alkyl, or arylalkyl can be prepared by this method. When Y₁≠Y₂, mixtures of diastereoisomers of formula Ia are obtained.

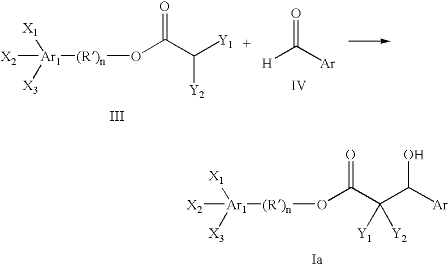

An alternative approach to the synthesis of formula Ia wherein Y₁=alkyl, aryl, arylalkyl, or cyano and Y₂=H is the boron-mediated aldol reaction of ester III (Y₁=alkyl, alkoxyalkyl, aryl, or arylalkyl and Y₂=H) and aldehyde IV (T. Inoue et al, 2002, J. Org. Chem 67, 5250-5256). Thus, when the boron enolate of III is formed at low temperature (typically −78° C.) with an amine base such as triethylamine and c-hex₂BOTf (dicyclohexylboron triflate) in an inert solvent such as dichloromethane, and then reacted with aldehyde IV, the anti-aldol product I (Y₂=H) is formed stereoselectively: 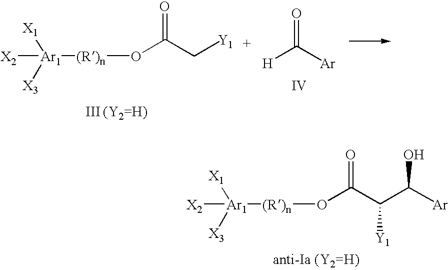
Conversely, when the boron enolate of III is formed at low temperature (typically −78° C.) with an amine base such as diisopropylethylamine and n-bu₂BOTf (di-n-butylboron triflate) in an inert solvent such as dichloromethane, and then reacted with aldehyde IV, the syn-aldol product is obtained stereoselectively: 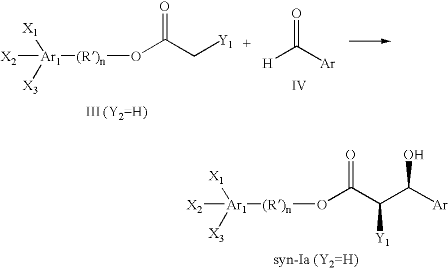
The syn-products are generally the more biologically active fungicides.

Compositions of formula Ia wherein Y₁=alkylthioalkyl, arylthioalkyl and Y₂=H are prepared by the Michael addition of a substituted thiol of formula VI (where R₁ is alkyl or aryl, the term “aryl” including heteroaryl as noted above) to an acrylate of formula V: 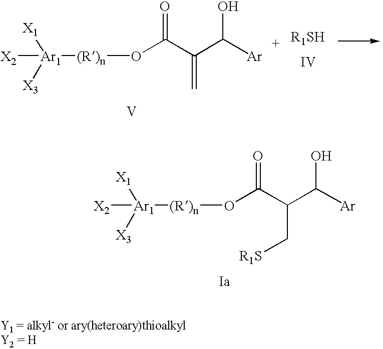
The Michael addition is generally done in the presence of an organic base (5-75 mol %) such as DABCO (1,4-diazabicyclo[2.2.2]octane) in organic solvents such as dioxane or tetrahydrofuran at 20-80° C.

Likewise, compositions of the formula Ia wherein Y₁=(substituted)aminoalkyl, or N-heterocycloalkyl and Y₂=H are prepared by the Michael addition of a (substituted)amine of formula VII (where R₂ and R₃ are each taken individually from alkyl; haloalkyl; aryl (including heteroaryl) optionally substituted with halogen, alkyl, alkynyl, haloalkyl, alkoxy, alkylthio, haloalkoxy, cyano, nitro; or R₂ and R₃ taken together to form a 5 or 6-member heterocycle containing 2-5 C-atoms, 0-1 O-atom, 0-1 S-atom, and 1-3 N-atoms) to acrylate of formula V: 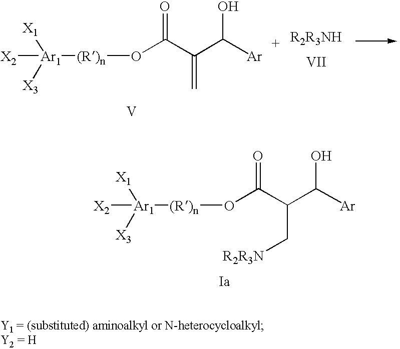
The Michael addition of a (substituted)amine can be done with/without a catalyst such as DMAP (4-dimethylaminopyridine) in an inert solvent such as THF. The Michael addition of a nitrogen-containing heterocycle is conveniently carried out using potassium carbonate in a solvent such as acetonitrile at 20-50° C.

Acrylates V are conveniently prepared using the Baylis-Hillman reaction of an unsubstituted acrylate ester VIII and aldehyde IV:

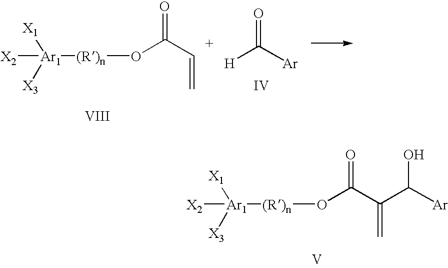

The Baylis-Hillman reaction is carried out in accordance with known techniques, or variations thereof which will be apparent to those skilled in the art (See, e.g., D. Basavaiah et al., “The Baylis-Hillman Reaction: A Novel Carbon-Carbon Bond Forming Reaction, Tetrahyedron 52, 8001-80062 (1996); see also U.S. Pat. No. 5,936,127). In general the reaction is carried out in the presence of a tertiary amine base such as DABCO (1,4-diazabicyclo[2.2.2]octane), trimethylamine, or DMAP (4-dimethylaminopyridine) with or without solvent or combinations thereof such as water, 1,4-dioxane, tetrahydrofuran, formamide, alcohol.

Compositions with the generic structure of formula II may be prepared by oxidation of the corresponding alcohol of formula Ia: 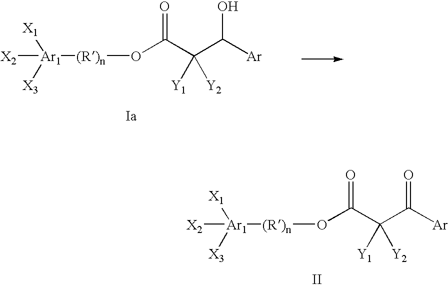
The oxidation is conveniently done using the Dess-Martin periodinane (D. B. Dess and J. C. Martin 1991, J. Amer. Chem. Soc. 113, 7277; R. E. Ireland and L. Lin 1993, J. Org. Chem. 58, 2899) in dichloromethane at low temperatures, usually −78° C.

Compositions that are especially useful for the control of fungal pathogens are those in which:
Ar=heteroaryl, especially 2- or 3-pyridyl; 2- or 5-thiazolyl;
Ar₁=phenyl;
X₁, X₂, and X₃ taken individually from H, halogen, alkyl, haloalkyl, alkoxy, haloalkoxy; aryl or aryloxy or aryloxyalkyl, optionally substituted with halogen, alkyl, haloalkyl, alkoxy, haloalkoxy, cyano;
R′ is CH₂.
n is 0 or 1.

Exemplary compounds. Examples of compounds of the present invention include, but are not limited to, the following:

| Compound | | |
|---|---|---|
| Number | Structure | Chemical Name |
| 1 | 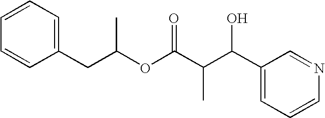 | 2-Phenyl-1-methylethyl β-hydroxy-α-methyl-3- pyridinepropanoate |
| 2 | 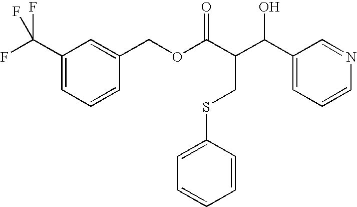 | 3-(Trifluoromethyl)phenylmethyl β-hydroxy-α- phenylthiomethyl-3-pyridinepropanoate |
| 3 | 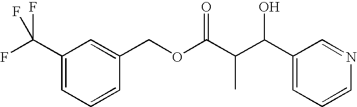 | 3-(Trifluoromethyl)phenylmethyl β-hydroxy-α- methyl-3-pyridinepropanoate |
| 4 | 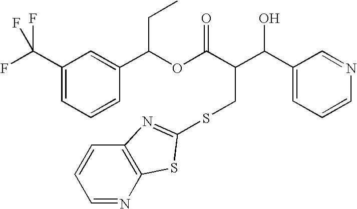 | 1-(3-Trifluoromethylphenyl)propyl β-hydroxy-α- {2-(thiazolo[5,4-b]pyridine)thiomethyl}-3- pyridinepropanoate |
| 5 | 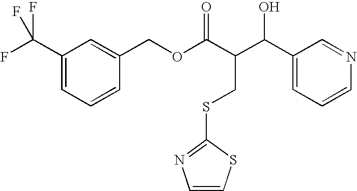 | 3-(Trifluoromethyl)phenylmethyl β-hydroxy-α- (2-thiazolylthiomethyl)-3-pyridinepropanoate |
| 6 | 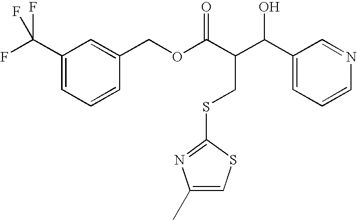 | 3-(Trifluoromethyl)phenylmethyl βhydroxy-α- (4-methyl-2-thiazolylthiomethyl)-3- pyridinepropanoate |
| 7 | 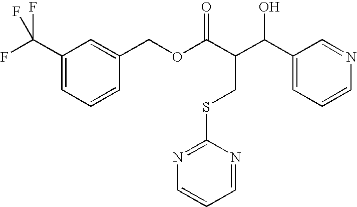 | 3-(Trifluoromethyl)phenylmethyl β-hydroxy-α- (2-pyrimidinylthiomethyl)-3-pyridinepropanoate |
| 8 | 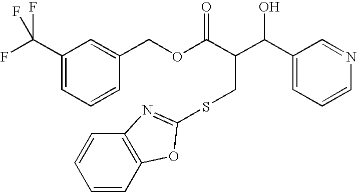 | 3-(Trifluoromethyl)phenylmethyl β-hydroxy-α- (2-benzoxazolylthiomethyl)-3-pyridinepropanoate |
| 9 | 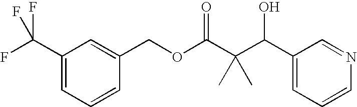 | 3-(Trifluoromethyl)phenylmethyl β-hydroxy-α,α- dimethyl-3-pyridinepropanoate |
| 10 | 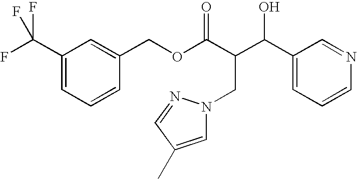 | 3-(Trifluoromethyl)phenyimethyl β-hydroxy-α- (4-methyl-1-pyrazolylmethyl)-3- pyridinepropanoate |
| 11 | 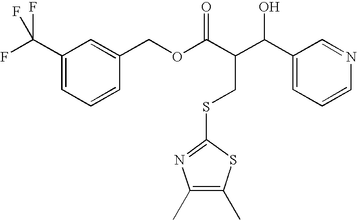 | 3-(Trifluoromethyl)phenylmethyl β-hydroxy-α- (4,5-dimethyi-2-thiazoiylthiomethyl)-3- pyridinepropanoate |
| 12 | 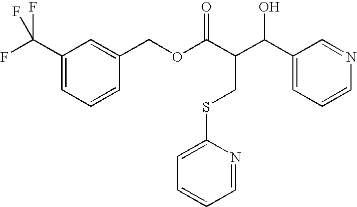 | 3-(Trifluoromethyl)phenylmethyl β-hydroxy-α- (2-pyridylthiomethyl)-3-pyridinepropanoate |
| 13 | 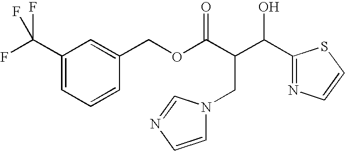 | 3-(Trifluoromethyl)phenylmethyl β-hydroxy-α- (1-imidazolylmethyl)-2-thiazolepropanoate |
| 14 | 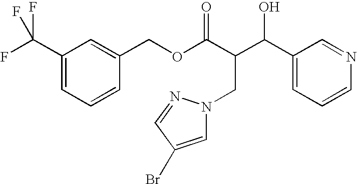 | 3-(Trifluoromethyl)phenylmethyl β-hydroxy-α- (4-bromo-1-pyrazolylmethyl)-3- pyridinepropanoate |
| 15 | 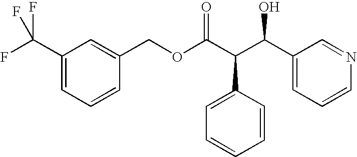 | 3-(Trifluoromethyl)phenylmethyl syn-β-hydroxy-α- phenyl-3-pyridinepropanoate |
| 16 | 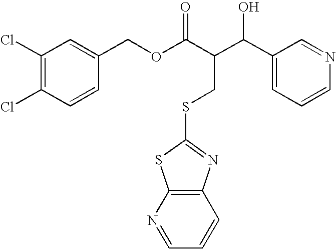 | 3,4-Dichlorophenylmethyl β-hydroxy-α- {2-(thiazolo[5,4-b]pyridine)thiomethyl}-3- pyridinepropanoate |
| 17 | 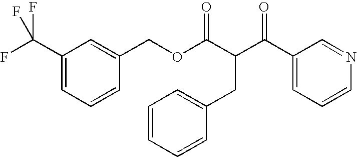 | 3-(Trifluoromethyl)phenytmethyl β-oxo-α- phenylmethyl-3-pyridinepropanoate |
| 18 | 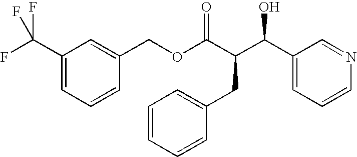 | 3-(Trifluoromethyl)phenylmethyl syn-β-hydroxy-α- phenylmethyl-3-pyridinepropanoate |
| 19 | 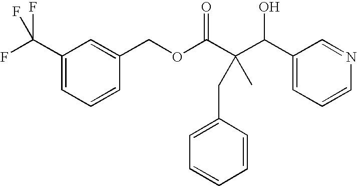 | 3-(Trifluoromethyi)phenylmethyl β-hydroxy-α- methyl-α-phenylmethyl-3-pyridinepropanoate |
| 20 | 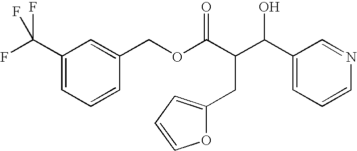 | 3-(Trifluoromethyl)phenylmethyl β-hydroxy-α- 2-furylmethyl-3-pyridinepropanoate |
| 21 | 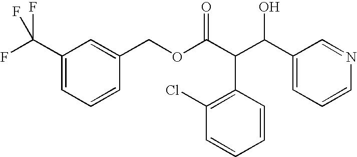 | 3-(Trifluoromethyl)phenylmethyl β-hydroxy-α- 2-chlorophenyl-3-pyridinepropanoate |
| 22 | 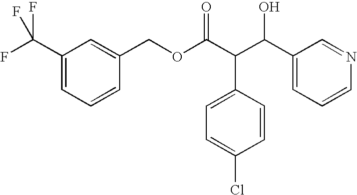 | 3-(Trifluoromethyl)phenylmethyl β-hydroxy-α- 4-chlorophenyl-3-pyridinepropanoate |
| 23 | 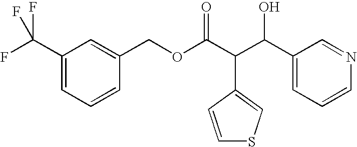 | 3-(Trifluoromethyl)phenylrnethyl β-hydroxy-α- (3-thienyl)-3-pyridinepropanoate |
| 24 | 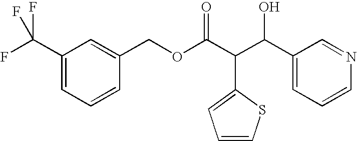 | 3-(Trifluoromethyl)phenylmethyl β-hydroxy-α- (2-thienyl)-3-pyridinepropanoate |
| 25 | 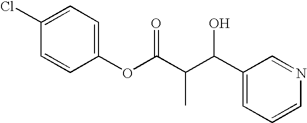 | 4-Chlorophenyl β-hydroxy-α-methyl-3- pyridinepropanoate |
| 26 | 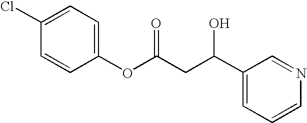 | 4-Chlorophenyl β-hydroxy-3-pyndinepropanoate |
| 27 | 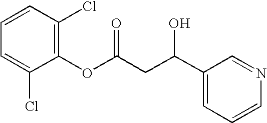 | 2,6-Dichlorophenyl β-hydroxy-3-pyridine- propanoate |
| 28 | 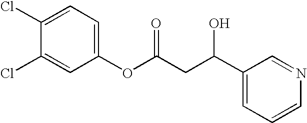 | 3,4-Dichlorophenyl β-hydroxy-3- pyridinepropanoate |
| 29 | 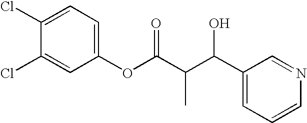 | 3,4-Dichlorophenyl β-hydroxy-α-methyl-3- pyridinepropanoate |
| 30 | 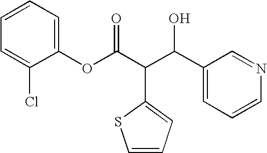 | 2-Chlorophenyl β-hydroxy-α-(2-thienyl)-3- pyridinepropanoate |
| 31 | 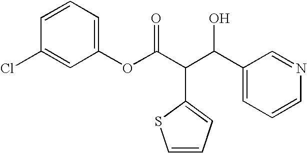 | 3-Chlorophenyl β-hydroxy-α-(2-thienyl)-3- pyridinepropanoate |
| 32 | 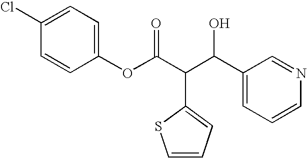 | 4-Chlorophenyl β-hydroxy-α-(2-thienyl)-3- pyridinepropanoate |
| 33 | 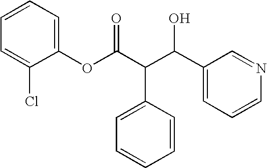 | 2-Chlorophenyl β-hydroxy-α-phenyl-3- pyridinepropanoate |
| 34 | 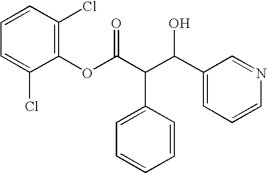 | 2,6-Dichlorophenyl β-hydroxy-α-phenyl-3- pyridinepropanoate |
| 35 | 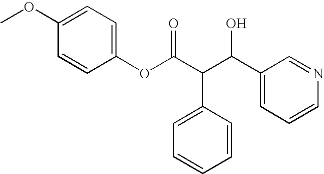 | 4-Methoxyphenyl β-hydroxy-α-phenyl-3- pyridinepropanoate |
| 36 | 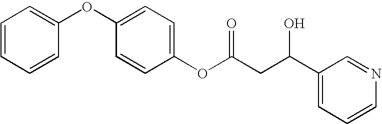 | 4-Phenoxyphenyl β-hydroxy-3-pyridine- propanoate |
| 37 | 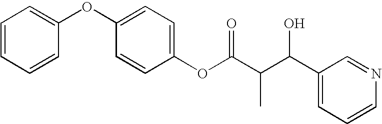 | 4-Phenoxyphenyl β-hydroxy-α-methyl-3- pyridinepropanoate |
| 38 | 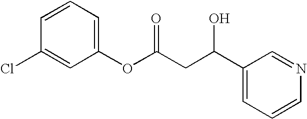 | 3-Chlorophenyl β-hydroxy-3-pyridinepropanoate |
| 39 | 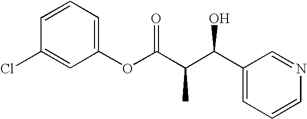 | 3-Chlorophenyl syn-β-hydroxy-α-methyl-3- pyridinepropanoate |
| 40 | 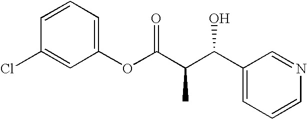 | 3-Chlorophenyl anti-β-hydroxy-α-methyl-3- pyridinepropanoate |
| 41 | 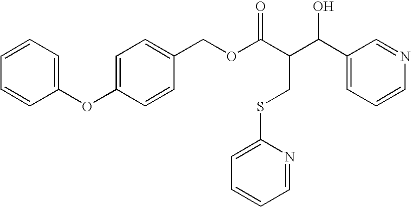 | 4-Phenoxyphenylmethyl β-hydroxy-α- (2-pyridylthiomethyl)-3-pyridinepropanoate |
| 42 | 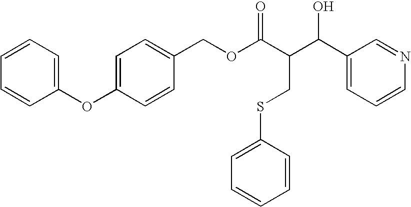 | 4-Phenoxyphenylmethyl β-hydroxy-α- phenylthiomethyl-3-pyridinepropanoate |
| 43 | 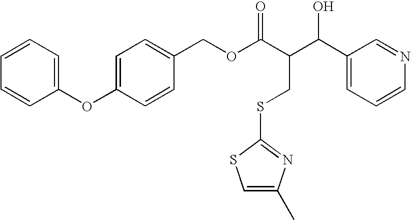 | 4-Phenoxyphenylmethyl β-hydroxy-α- (4-methyl-2-thiazolylthiomethyl)-3- pyridinepropanoate |
| 44 | 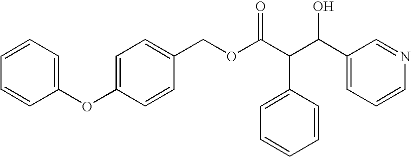 | 4-Phenoxyphenylmethyl β-hydroxy-α- phenyl-3-pyridinepropanoate |
| 45 | 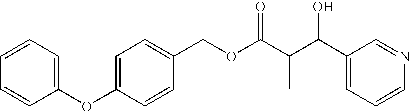 | 4-Phenoxyphenylmethyl β-hydroxy-α- methyl-3-pyridnepropanoate |
| 46 | 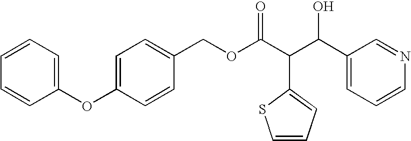 | 4-Phenoxyphenylmethyl β-hydroxy-α- (2-thienyl)-3-pyridinepropanoate |
| 47 | 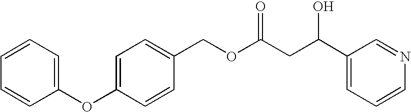 | 4-Phenoxyphenylmethyl β-hydroxy-3- pyridinepropanoate |
| 48 | 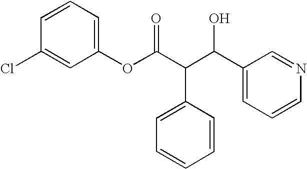 | 3-Chlorophenyl β-hydroxy-α-phenyl-3- pyridinepropanoate |
| 49 | 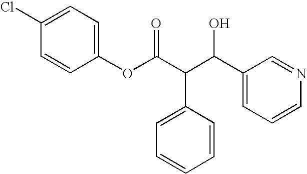 | 4-Chlorophenyl β-oxo-α-phenyl-3- pyridinepropanoate |
| 50 | 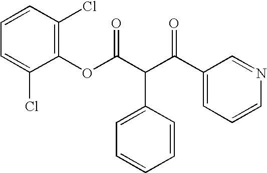 | 2,6-Dichlorophenyl β-oxo-α-phenyl-3- pyridinepropanoate |
| 51 | 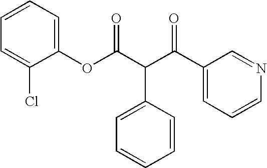 | 2-Chlorophenyl β-oxo-α-phenyl-3- pyridinepropanoate |
| 52 | 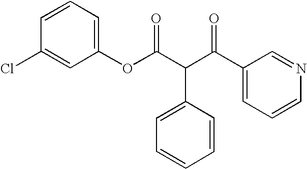 | 3-Chlorophenyl β-oxo-α-phenyl-3- pyridinepropanoate |
| 53 | 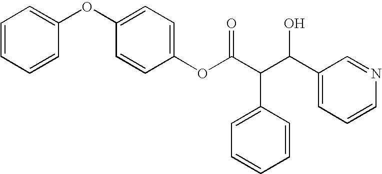 | 4-Phenoxyphenyl β-hydroxy-α-phenyl-3- pyridinepropanoate |
| 54 | 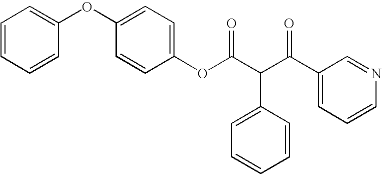 | 4-Phenoxyphenyl β-oxo-α-phenyl-3- pyridinepropanoate |
| 55 | 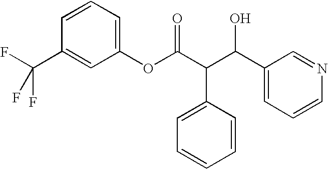 | 3-Trifluoromethylphenyl β-hydroxy-α-phenyl-3- pyridinepropanoate |
| 56 | 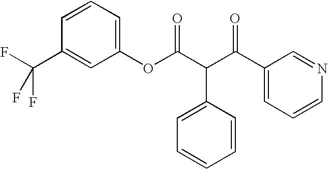 | 3-Trifluoromethylphenyl β-oxo-α-phenyl-3- pyridinepropanoate |
| 57 | 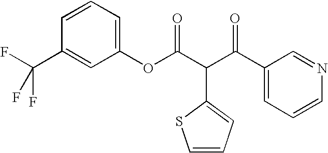 | 3-Trifluoromethylphenyl β-oxo-α-(2-thienyl)-3- pyridinepropanoate |
| 58 | 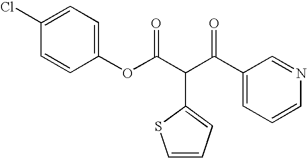 | 4-Chlorophenyl β-oxo-α-(2-thienyl)-3- pyridinepropanoate |
| 59 | 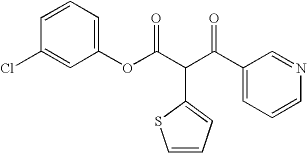 | 3-Chlorophenyl β-oxo-α-(2-thienyl)-3- pyridinepropanoate |
| 60 | 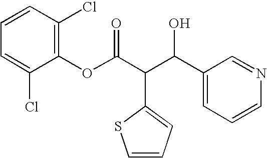 | 2,6-Dichlorophenyl β-hydroxy-α-(2-thienyl)-3- pyridinepropanoate |
| 61 | 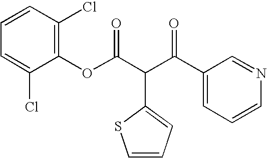 | 2,6-Dichlorophenyl β-oxo-α-(2-thienyl)-3- pyridinepropanoate |
| 62 | 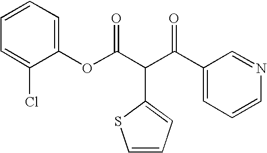 | 2-Chlorophenyl β-oxo-α-(2-thienyl)-3- pyridinepropanoate |
| 63 | 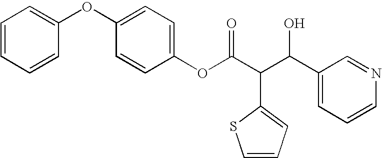 | 4-Phenoxyphenyl β-hydroxy-α-(2-thienyl)-3- pyridinepropanoate |
| 64 | 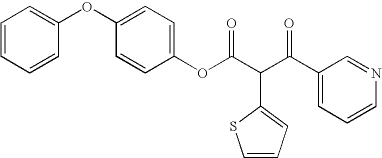 | 4-Phenoxyphenyl β-oxo-α-(2-thienyl)-3- pyridinepropanoate |
| 65 | 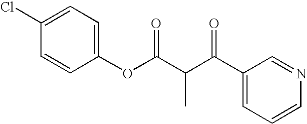 | 4-Chlorophenyl β-oxo-α-methyl-3- pyridinepropanoate |
| 66 | 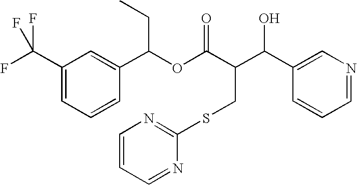 | 1-(3-Trifluoromethylphenyl)propyl β- hydroxy-α-(2-pyrimidinylthiomethyl)-3- pyridinepropanoate |
| 67 | 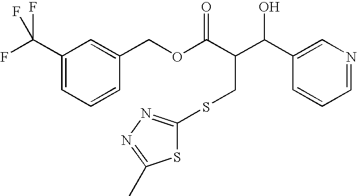 | 3-(Trifluoromethyl)phenylmethyl β-hydroxy-α-(5-methyl-1,3,4-thiadiazolyl-2- thiomethyl)-3-pyridinepropanoate |
| 68 | 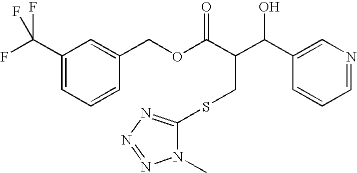 | 3-(Trifluoromethyl)phenylmethyl β-hydroxy-α-(1-methyl-1,2,3,4-tetrazolyl-5- thiomethyl)-3-pyridinepropanoate |
| 69 | 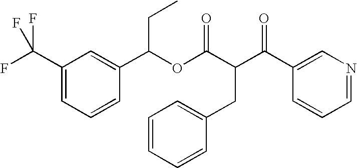 | 1-(3-Trifluoromethylphenyl)propyl β-oxo-α- phenylmethyi-3-pyridinepropanoate |
| 70 | 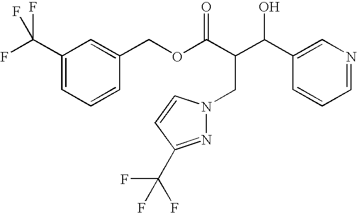 | 3-(Trifluoromethyl)phenylmethyl β-hydroxy-α-(3-trifluoromethyl-1- pyrazolylmethyl)-3-pyridinepropanoate |
| 71 | 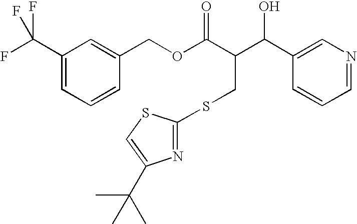 | 3-(Trifluoromethyl)phenylmethyl β-hydroxy-α-[4-(1,1-dimethyl-ethyl)-2- thiazolylthiomethyl]-3-pyridinepropanoate |
| 72 | 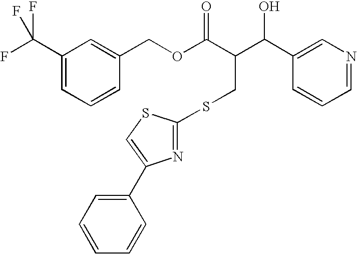 | 3-(Trifluoromethyl)phenylmethyl β-hydroxy-α-(4-phenyl-2- thiazolylthiomethyl)-3-pyridine-propanoate |
| 73 | 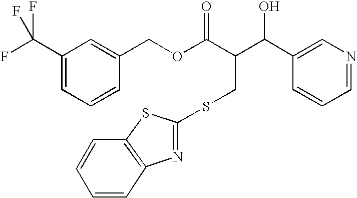 | 3-(Trifluoromethyl)phenylmethyl β-hydroxy-α-(2-benzthiazolylthiomethyl)-3- pyridine-propanoate |
| 74 | 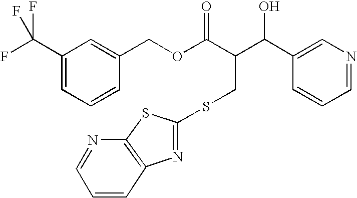 | 3-(Trifluoromethyl)phenylmethyl β-hydroxy-α-{2-(thiazolo[5,4-b]- pyridine)thiomethyl}-3-pyridinepropanoate |
| 75 | 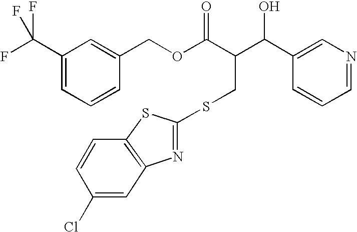 | 3-(Trifluoromethyl)phenylmethyl β-hydroxy-α-(5-chloro-2- benzthiazolylthiomethyl)-3-pyridine- propanoate |
| 76 | 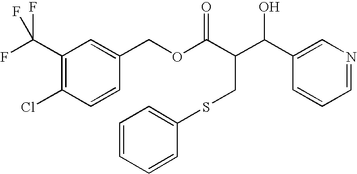 | 4-Chloro-3-(trifluoromethyl)phenylmethyl β-hydroxy-α-{phenylthiomethyl}-3- pyridinepropanoate |
| 77 | 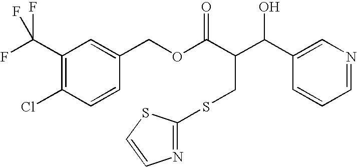 | 4-Chloro-3-(trifluoromethyl)phenylmethyl β-hydroxy-α-(2-thiazolylthiomethyl)-3- pyridinepropanoate |
| 78 | 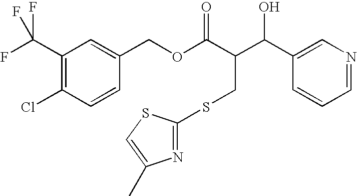 | Chloro-3-(trifluoromethyl)phenylmethyl β-hydroxy-α-(4-methyl-2- thiazolylthiomethyl)-3-pyridinepropanoate |
| 79 | 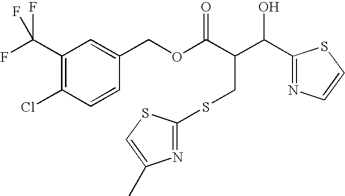 | 4-Chloro-3-(trifluoromethyl)phenylmethyl β-hydroxy-α-(4-methyl-2- thiazolylthiomethyl)-2-thiazolepropanoate |
| 80 | 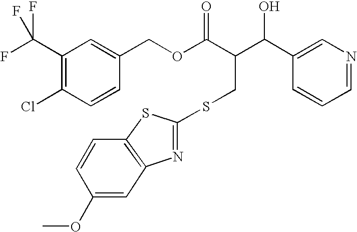 | 4-Chloro-3-(trifluoromethyl)phenylmethyl β-hydroxy-α-(5-methoxy-2- benzthiazolylthiomethyl)-3- pyridinepropanoate |
| 81 | 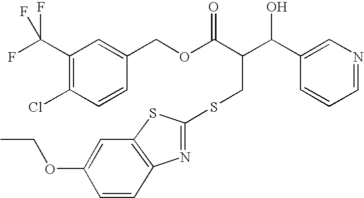 | 4-Chloro-3-(trifluoromethyl)phenylmethyl β-hydroxy-α-(6-ethoxy-2-benzthiazolyl- thiomethyl)-3-pyridinepropanoate |
| 82 | 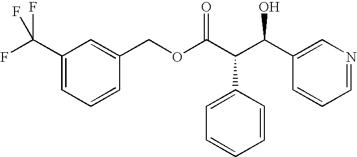 | 3-(Trifluoromethyl)phenylmethyl anti-β- hydroxy-α-phenyl-3-pyridinepropanoate |
| 83 | 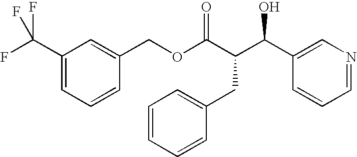 | 3-(Trifluoromethyl)phenylmethyl anti-β- hydroxy-α-phenylmethyl-3- pyridinepropanoate |
| 84 | 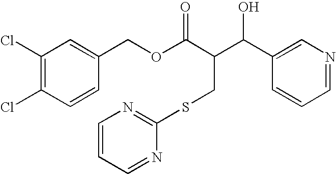 | 3,4-Dichlorophenylmethyl β-hydroxy-α-(2- pynmidinylthiomethyl)-3- pyridinepropanoate |
| 85 | 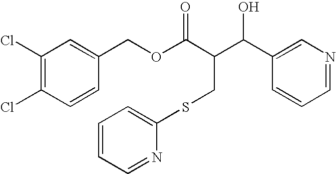 | 3,4-Dichlorophenylmethyl β-hydroxy-α-(2- pyridinylthiomethyl)-3-pyridinepropanoate |
| 86 | 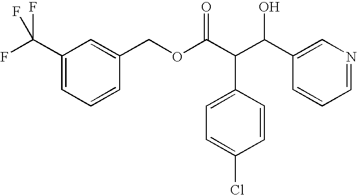 | 3-(Trifluoromethyl)phenylmethyl β- hydroxy-α-(4-chlorophenyl)-3- pyridinepropanoate |
| 87 | 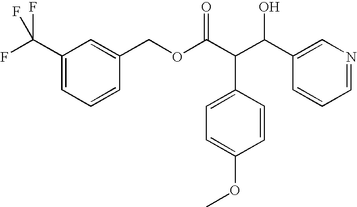 | 3-(Trifluoromethyl)phenylmethyl β- hydroxy-α-(4-methoxyphenyl)-3- pyridinepropanoate |
| 88 | 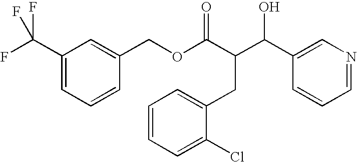 | 3-(Trifluoromethyl)phenylmethyl β- hydroxy-α-(2-chlorophenylmethyl)-3- pyridinepropanoate |
| 89 | 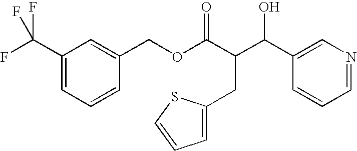 | 3-(Trifluoromethyl)phenylmethyl β- hydroxy-α-(2-thienylmethyl)-3- pyridinepropanoate |
| 90 | 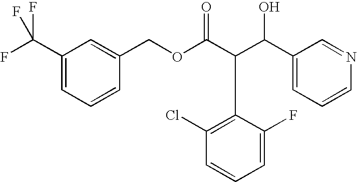 | 3-(Trifluoromethyl)phenylmethyl β- hydroxy-α-(2-chloro-6-fluorophenyl)-3- pyridinepropanoate |
| 91 | 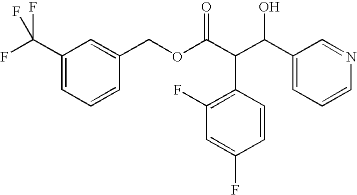 | 3-(Trifluoromethyl)phenylmethyl β- hydroxy-α-(2,4-difluorophenyl)-3- pyridinepropanoate |
| 92 | 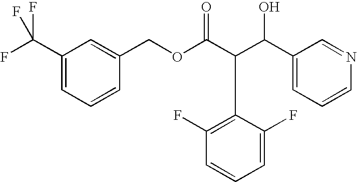 | 3-(Trifluoromethyl)phenylmethyl β- hydroxy-α-(2,6-difluorophenyl)-3- pyridinepropanoate |

Salts. The compounds described herein and, optionally, all their isomers may be obtained in the form of their salts. Because some of the compounds I have a basic center they can, for example, form acid addition salts. Said acid addition salts are, for example, formed with mineral acids, typically sulfuric acid, a phosphoric acid or a hydrogen halide, with organic carboxylic acids, typically acetic acid, oxalic acid, malonic acid, maleic acid, fumaric acid or phthalic acid, with hydroxycarboxylic acids, typically ascorbic acid, lactic acid, malic acid, tartaric acid or citric acid, or with benzoic acid, or with organic sulfonic acids, typically methanesulfonic acid or p-toluenesulfonic acid. Together with at least one acidic group, the compounds of formula I can also form salts with bases. Suitable salts with bases are, for example, metal salts, typically alkali metal salts; or alkaline earth metal salts, e.g. sodium salts, potassium salts or magnesium salts, or salts with ammonia or an organic amine, e.g. morpholine, piperidine, pyrrolidine, a mono-, di- or trialkylamine, typically ethylamine, diethylamine, triethylamine or dimethylpropylamine, or a mono-, di- or trihydroxyalkylamine, typically mono-, di- or triethanolamine. Where appropriate, the formation of corresponding internal salts is also possible. Within the scope of this invention, agrochemical or pharmaceutically acceptable salts are preferred.

3. Agrochemical compositions and use. Active compounds of the present invention can be used to prepare agrochemical compositions and used to control fungi in like manner as other antifungal compounds. See, e.g., U.S. Pat. No. 6,617,330; see also U.S. Pat. Nos. 6,616,952; 6,569,875; 6,541,500, and 6,506,794.

Active compounds described herein can be used for protecting plants against diseases that are caused by fungi. For the purposes herein, oomycetes shall be considered fungi. The active compounds can be used in the agricultural sector and related fields as active ingredients for controlling plant pests. The active compounds can be used to inhibit or destroy the pests that occur on plants or parts of plants (fruit, blossoms, leaves, stems, tubers, roots) of different crops of useful plants, optionally while at the same time protecting also those parts of the plants that grow later e.g. from phytopathogenic micro-organisms.

Active compounds may be used as dressing agents for the treatment of plant propagation material, in particular of seeds (fruit, tubers, grains) and plant cuttings (e.g. rice), for the protection against fungal infections as well as against phytopathogenic fungi occurring in the soil.

The active compounds may be used, for example, against the phytopathogenic fungi of the following classes: Fungi imperfecti (e.g. Botrytis, Pyricularia, Heiminthosporium, Fusarium, Septoria, Cercospora and Alternaria) and Basidiomycetes (e.g. Rhizoctonia, Hemileia, Puccinia). Additionally, they may also be used against the Ascomycetes classes (e.g. Venturia and Erysiphe, Podosphaera, Monilinia, Uncinula) and of the Oomycetes classes (e.g. Phytophthora, Pythium, Plasmopara). Specific examples of fungi that may be treated include, but are not limited to, Septoria tritici, Stagnospora nodorum, Phytophthora infestans, Ustilago maydis, Botrytis cinerea and Erysiphe graminis.

Target crops to be protected with active compounds and compositions of the invention typically comprise the following species of plants: cereal (wheat, barley, rye, oat, rice, maize, sorghum and related species); beet (sugar beet and fodder beet); pomes, drupes and soft fruit (apples, pears, plums, peaches, almonds, cherries, strawberries, raspberries and blackberries); leguminous plants (beans, lentils, peas, soybeans); oil plants (rape, mustard, poppy, olives, sunflowers, coconut, castor oil plants, cocoa beans, groundnuts); cucumber plants (pumpkins, cucumbers, melons); fiber plants (cotton, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocado, cinnamon, camphor) or plants such as tobacco, nuts, coffee, eggplants, sugar cane, tea, pepper, vines including grape-bearing vines, hops, bananas, turf and natural rubber plants, as well as ornamentals (flowers, shrubs, broad-leafed trees and evergreens, such as conifers). This list does not represent any limitation.

The active compounds can be used in the form of compositions and can be applied to the crop area or plant to be treated, simultaneously or in succession with further compounds. These further compounds can be e.g. fertilizers or micronutrient donors or other preparations which influence the growth of plants. They can also be selective herbicides as well as insecticides, fungicides, bactericides, nematicides, molluscicides, plant growth regulators, plant activators or mixtures of several of these preparations, if desired together with further carriers, surfactants or application promoting adjuvants customarily employed in the art of formulation.

The active compounds can be mixed with other fungicides, resulting in some cases in unexpected synergistic activities.

Mixing components which are particularly preferred are azoles such as azaconazole, bitertanol, propiconazole, difenoconazole, diniconazole, cyproconazole, epoxiconazole, fluquinconazole, flusilazole, flutriafol, hexaconazole, imazalil, imibenconazole, ipconazole, prothioconazole, tebuconazole, tetraconazole, fenbuconazole, metconazole, myclobutanil, perfurazoate, penconazole, bromuconazole, pyrifenox, prochloraz, triadimefon, triadimenol, triflumizole or triticonazole; pyrimidinyl carbinoles such as ancymidol, fenarimol or nuarimol; 2-amino-pyrimidine such as bupirimate, dimethirimol or ethirimol; morpholines such as dodemorph, fenpropidin, fenpropimorph, spiroxamin or tridemorph; anilinopyrimidines such as cyprodinil, pyrimethanil or mepanipyrim; pyrroles such as fenpiclonil or fludioxonil; phenylamides such as benalaxyl, furalaxyl, metalaxyl, R-metalaxyl, ofurace or oxadixyl; benzimidazoles such as benomyl, carbendazim, debacarb, fuberidazole or thiabendazole; dicarboximides such as chlozolinate, dichlozoline, iprodine, myclozoline, procymidone or vinclozolin; carboxamides such as carboxin, fenfuram, flutolanil, mepronil, oxycarboxin or thifluzamide; guanidines such as guazatine, dodine or iminoctadine; strobilurines such as azoxystrobin, kresoxim-methyl, metominostrobin, SSF-129, flucoxastrobin, pyraclostrobin, methyl 2 [(2-trifluoromethyl)-pyrid-6-yloxymethyl]-3-methoxy-acrylate or 2-[{.alpha.[(.alpha.-methyl-3-trifluoromethyl-benzyl)imino]-oxy}-o-tolyl]-glyoxylic acid-methylester-O-methyloxime (trifloxystrobin); dithiocarbamates such as ferbam, mancozeb, maneb, metiram, propineb, thiram, zineb or ziram; N-halomethylthio-dicarboximides such as captafol, captan, dichlofluanid, fluoromide, folpet or tolyfluanid; copper compounds such as Bordeaux mixture, copper hydroxide, copper oxychloride, copper sulfate, cuprous oxide, mancopper or oxine-copper; nitrophenol derivatives such as dinocap or nitrothal-isopropyl; organo phosphorous derivatives such as edifenphos, iprobenphos, isoprothiolane, phosdiphen, pyrazophos or toclofos-methyl; and other compounds of diverse structures such as acibenzolar-S-methyl, harpin, anilazine, blasticidin-S, chinomethionat, chloroneb, chlorothalonil, cymoxanil, dichlone, diclomezine, dicloran, diethofencarb, dimethomorph, dithianon, etridiazole, famoxadone, fenamidone, fentin, ferimzone, fluazinam, flusulfamide, fenhexamid, fosetyl-aluminium, hymexazol, kasugamycin, methasulfocarb, pencycuron, phthalide, polyoxins, probenazole, propamocarb, pyroquilon, quinoxyfen, quintozene, sulfur, triazoxide, tricyclazole, triforine, validamycin, (S)-5-methyl-2-methylthio-5-phenyl-3-phenylamino-3,5-di-hydroimidazol-4-one (RPA 407213), 3,5-dichloro-N-(3-chloro-1-ethyl-1-methyl-2-oxopropyl)-4-methylbenzamide (RH-7281), N-allyl-4,5-dimethyl-2-trimethylsilylthiophene-3-carboxamide (MON 65500), 4-chloro-4-cyano-N,N-dimethyl-5-p-tolylimidazole-1-sulfon-amide (IKF-916), N-(1-cyano-1,2-dimethylpropyl)-2-(2,4-dichlorophenoxy)-propionamide (AC 382042) or iprovalicarb (SZX 722).

Suitable carriers and adjuvants can be solid or liquid and are substances useful in formulation technology, e.g. natural or regenerated mineral substances, solvents, dispersants, wetting agents, tackifiers, thickeners, binders or fertilizers.

A preferred method of applying an active compound of the invention, or an agrochemical composition which contains at least one of said compounds, is foliar application. The frequency of application and the rate of application will depend on the risk of infestation by the corresponding pathogen. However, the active compounds can also penetrate the plant through the roots via the soil (systemic action) by drenching the locus of the plant with a liquid formulation, or by applying the compounds in solid form to the soil, e.g. in granular form (soil application). In crops of water such as rice, such granulates can be applied to the flooded rice field. The active compounds may also be applied to seeds (coating) by impregnating the seeds or tubers either with a liquid formulation of the fungicide or coating them with a solid formulation.

The term locus as used herein is intended to embrace the fields on which the treated crop plants are growing, or where the seeds of cultivated plants are sown, or the place where the seed will be placed into the soil. The term seed is intended to embrace plant propagating material such as cuttings, seedlings, seeds, and germinated or soaked seeds.

The active compounds are used in unmodified form or, preferably, together with the adjuvants conventionally employed in the art of formulation. To this end they are conveniently formulated in known manner to emulsifiable concentrates, coatable pastes, directly sprayable or dilutable solutions, dilute emulsions, wettable powders, soluble powders, dusts, granulates, and also encapsulations e.g. in polymeric substances. As with the type of the compositions, the methods of application, such as spraying, atomizing, dusting, scattering, coating or pouring, are chosen in accordance with the intended objectives and the prevailing circumstances.

Advantageous rates of application are normally from 5 g to 2 kg of active ingredient (a.i.) per hectare (ha), preferably from 10 g to 1 kg a.i./ha, most preferably from 20 g to 600 g a.i./ha. When used as seed drenching agent, convenient dosages are from 10 mg to 1 g of active substance per kg of seeds.

The formulation, i.e. the compositions containing the compound of formula I and, if desired, a solid or liquid adjuvant, are prepared in known manner, typically by intimately mixing and/or grinding the compound with extenders, e.g. solvents, solid carriers and, optionally, surface active compounds (surfactants).

Suitable carriers and adjuvants may be solid or liquid and correspond to the substances ordinarily employed in formulation technology, such as, e.g. natural or regenerated mineral substances, solvents, dispersants, wetting agents, tackifiers, thickeners binding agents or fertilizers. Such carriers are for example described in WO 97/33890.

Further surfactants customarily employed in the art of formulation are known to the expert or can be found in the relevant literature.

The agrochemical formulations will usually contain from 0.1 to 99% by weight, preferably from 0.1 to 95% by weight, of the compound of formula 1,99.9 to 1% by weight, preferably 99.8 to 5% by weight, of a solid or liquid adjuvant, and from 0 to 25% by weight, preferably from 0.1 to 25% by weight, of a surfactant.

Whereas it is preferred to formulate commercial products as concentrates, the end user will normally use dilute formulations.

The compositions may also contain further adjuvants such as stabilizers, antifoams, viscosity regulators, binders or tackifiers as well as fertilizers, micronutrient donors or other formulations for obtaining special effects.

4. Technical materials. The compounds and combinations of the present invention may also be used in the area of controlling fungal infection (particularly by mold and mildew) of technical materials, including protecting technical material against attack of fungi and reducing or eradicating fungal infection of technical materials after such infection has occurred. Technical materials include but are not limited to organic and inorganic materials wood, paper, leather, natural and synthetic fibers, composites thereof such as particle board, plywood, wall-board and the like, woven and non-woven fabrics, construction surfaces and materials, cooling and heating system surfaces and materials, ventilation and air conditioning system surfaces and materials, and the like. The compounds and combinations according the present invention can be applied to such materials or surfaces in an amount effective to inhibit or prevent disadvantageous effects such as decay, discoloration or mold in like manner as described above. Structures and dwellings constructed using or incorporating technical materials in which such compounds or combinations have been applied are likewise protected against attack by fungi.

5. Pharmaceutical uses. In addition to the foregoing, active compounds of the present invention can be used in the treatment of fungal infections of human and animal subjects (including but not limited to horses, cattle, sheep, dogs, cats, etc.) for medical and veterinary purposes. Examples of such infections include but are not limited to ailments such as Onychomycosis, sporotichosis, hoof rot, jungle rot, Pseudallescheria boydii, scopulariopsis or athletes foot, sometimes generally referred to as “white-line” disease, as well as fungal infections in immunocomprised patients such as AIDS patients and transplant patients. Thus, fungal infections may be of skin or of keratinaceous material such as hair, hooves, or nails, as well as systemic infections such as those caused by Candida spp., Cryptococcus neoformans, and Aspergillus spp., such as as in pulmonary aspergillosis and Pneumocystis carinii pneumonia. Active compounds as described herein may be combined with a pharmaceutically acceptable carrier and administered or applied to such subjects or infections (e.g., topically, parenterally) in an amount effective to treat the infection in accordance with known techniques, as (for example) described in U.S. Pat. Nos. 6,680,073; 6,673,842; 6,664,292; 6,613,738; 6,423,519; 6,413,444; 6,403,063; and 60,42,845; the disclosures of which applicants specifically intend be incoroporated by reference herein in their entirety.

“Pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically-acceptable carrier” as used herein means a pharmaceutically-acceptable material, composition or vehicle, such as a liquid or solid filler, diluent, excipient, solvent or encapsulating material, involved in carrying or transporting the subject peptidomimetic agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the active ingredient which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a peptide or peptidomimetic of the present invention with liquid carriers, or finely divided solid carriers, or both, and then, if necessary, shaping the product.

The ointments, pastes, creams and gels may contain, in addition to the active ingredient, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Pharmaceutical compositions of this invention suitable for parenteral administration comprise one or more active compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. These compositions may also contain adjuvants such as preservatives, wetting agents, emulsifying agents and dispersing agents. Prevention of the action of microorganisms may be ensured by the inclusion of various antibacterial and other antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The preparations of the present invention may be given by any suitable means of administration including orally, parenterally, topically, transdermally, rectally, etc. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Topical or parenteral administration is preferred.

“Parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

Actual dosage levels of the active ingredients in the pharmaceutical compositions of this invention may be varied so as to obtain an amount of the active ingredient which is effective to achieve the desired therapeutic response, e.g., antimycotic activity, for a particular patient, composition, and mode of administration, without being toxic to the patient. The selected dosage level will depend upon a variety of factors including the activity of the particular active compound employed, the route of administration, the time of administration, the rate of excretion of the particular active compound being employed, the duration of the treatment, other drugs, compounds and/or materials used in combination with the particular inhibitor employed, the age, sex, weight, condition, general health and prior medical history of the patient being treated, and like factors well known in the medical arts. A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved. As a general proposition, a dosage from about 0.01 or 0.1 to about 50, 100 or 200 mg/kg will have therapeutic efficacy, with all weights being calculated based upon the weight of the active compound, including the cases where a salt is employed.

The present invention is explained in greater detail in the following non-limiting Examples.

### EXAMPLE 1

### 3-(Trifluoromethyl)phenylmethyl syn-β-hydroxy-α-phenyl-3-pyridinepropanoate (Compound 15)

To a solution of 50 mg (0.17 mmol) of 3-(trifluoromethyl)phenylmethyl phenylacetate and 71 μL (53 mg, 0.41 mmol) of diisopropylethylamine in 2.5 mL of dichloromethane (DCM) at −78° C. under a nitrogen atmosphere was added 0.34 mL (0.34 mmol) of a 1.0M solution of dibutylboron triflate in DCM. The solution was stirred for 2.5 hrs, and then 20 mg (0.19 mmol) of 3-pyridinecarboxaldehyde was added. The reaction was stirred at −78° C. for an additional 2 hrs, and at 0° C. for 1 hr. The reaction mixture was quenched with saturated ammonium chloride, and extracted several times with diethyl ether. The combined ether extracts were washed with saturated sodium chloride and then dried (MgSO₄). Solvent was removed by rotoevaporation, and the crude product was purified by prep TLC to give 7.5 mg of 3-(trifluoromethyl)phenylmethyl syn-β-hydroxy-α-phenyl-3-pyridinepropanoate. ¹H NMR (CDCl₃): δ 3.92 (1H), 5.00 (1H), and 5.22 ppm (2H). MS m/z: 402.1 (M+H). The anti-isomer was prepared analogously except the boron enolate was prepared using dicyclohexylboron triflate and triethylamine.

### EXAMPLES 2-41

### Preparation of Additional β-Hydroxy-3-pyridinepropanoates

Compounds 18, 20-40, 44-48, 53, 55, 60, 63, 82, 83, and 86-92 above are prepared in essentially the same manner as described in Example 1 above. In some cases, mixtures of the syn- and anti-isomers were obtained.

### EXAMPLE 42

### 3-(Trifluoromethyl)phenylmethyl-β-hydroxy-α,α-dimethyl-3-pyridinepropanoate (Compound 9)

A solution of lithium diisopropylamide was freshly prepared by adding 1.25 mL (2.0 mmol) of n-butyllithium in hexane to 307 μL (222 mg, 2.2 mol) of diisopropylamine in 5 mL of anhydrous tetrahydrofuran (THF) at 0° C. and under a nitrogen atmosphere. After 30 min, the solution was cooled to −78° C., and 370 mg (1.5 mmol) of 3-(trifluoromethyl)phenylmethyl 2-methylpropanoate in 0.5 mL of THF was added dropwise. After an additional 2 hrs, 160 mg (1.5 mmol) of 3-pyridinecarboxaldehyde in 0.5 mL of THF was added dropwise. After another 1.5 hrs, the reaction was quenched with saturated ammonium chloride and warmed to room temperature. The THF was removed by rotoevaporation, and the residue was dissolved in DCM and water. The DCM fraction was washed sequentially with saturated sodium bicarbonate and saturated sodium chloride, and then dried (Na₂SO₄). Solvent was removed by rotoevaporation, and the residue was purified by prep TLC to give 23 mg (0.07 mmol) of 3-(trifluoromethyl)-phenylmethyl β-hydroxy-α,α-dimethyl-3-pyridinepropanoate. ¹H NMR (CDCl₃): δ 1.13 (3H), 1.19 (3H), 4.97 (1H), 5.20 (2H), and 8.43 ppm (2H). MS m/z: 354.1 (M+H).

### EXAMPLES 43-45

### Preparation of Additional β-hydroxy-3-pyridinepropanoates

Compounds 1, 3, and 19 above are prepared in essentially the same manner as described in Example 33 above.

### EXAMPLE 46

### 3-(Trifluoromethyl)phenylmethyl-β-hydroxy-α-(4-methyl-2-thiazolylthiomethyl)-3-pyridinepropanoate (Compound 6)

To a solution of 34 mg (0.1 mmol) of 3-(trifluoromethyl)phenylmethyl β-hydroxy-α-methylene-3-pyridinepropanoate and 26 mg (0.2 mmol) of 2-mercapto-4-methylthiazole in 2 mL of dioxane was added 6 mg (0.05 mmol) of DABCO. The reaction was heated at 70° C. overnight. Solvent was removed by rotoevaporation, and the residue was purified by prep HPLC to give 17 mg (0.04 mmol) of 3-(trifluoromethyl)phenylmethyl β-hydroxy-α-(4-methyl-2-thiazolylthiomethyl)-3-pyridinepropanoate. ¹H NMR (CDCl₃): δ MS m/z: XX (M+H).

### EXAMPLES 47-73

### Preparation of Additional β-Hydroxy-α-[(substituted)thiomethyl]-3-pyridinepropanoates

Compounds 2, 4, 5, 7, 8 11, 12, 16, 41-43, 66-68, 71-81, 84, and 85 above are prepared in essentially the same manner as described in Example 37 above.

### EXAMPLE 74

### 3-(Trifluoromethyl)phenylmethyl β-hydroxy-α-(4-methyl-1-pyrazolylmethyl)-3-pyridinepropanoate (Compound 10)

A mixture of 55 mg (0.16 mmol) of 3-(trifluoromethyl)phenylmethyl β-hydroxy-α-methylene-3-pyridinepropanoate, 24 μL (0.29 mmol) of 4-methylpyrazole, and 50 mg (0.33 mmol) of potassium carbonate in 2.5 mL of acetonitrile was shaken overnight at room temperature. The mixture was poured into diethyl ether and water. The ether layer was separated, washed with saturated sodium chloride, and dried (Na₂SO₄). The solvent was removed by rotoevaporation and the residue was purified by preparative TLC to give 18 mg (0.04 mmol) of 3-(trifluoromethyl)phenylmethyl β-hydroxy-α-(4-methyl-1-pyrazolylmethyl)-3-pyridinepropanoate. ¹H NMR (CDCl₃): δ 2.00 (3H), 3.28 (m) and 3.50 (m) (1H), 4.96 ppm (2H). MS m/z: 420.1 (M+H).

### EXAMPLES 75-77

### Preparation of Additional β-Hydroxy-α-[(substituted)aminomethyl]-3-pyridinepropanoates

Compounds 13, 14, and 70 above are prepared in essentially the same manner as described in Example 49 above.

### EXAMPLE 78

### 3-(Trifluoromethyl)phenylmethyl β-oxo-α-phenylmethyl-3-pyridinepropanoate (Compound 17)

A mixture of 35 mg (0.084 mmol) of 3-(trifluoromethyl)phenylmethyl β-hydroxy-α-phenylmethyl-3-pyridinepropanoate, prepared according to Example 1, and 107 mg (0.25 mmol) of the Dess-Martin reagent in 4 mL of DCM was stirred at room temperature for 72 hrs. The reaction mixture was poured into water and DCM, and the layers were separated. The DCM layer was washed with saturated sodium chloride and then dried (Na₂SO₄). Solvent was removed by rotoevaporation, and the residue was purified by preparative TLC to give 16 mg (0.038 mmol) of 3-(trifluoromethyl)phenylmethyl β-oxo-α-phenylmethyl-3-pyridinepropanoate. ¹H NMR (CDCl₃): δ 3.36 (d, 2H), 4.65 (t, 1H), 5.10 (s, 2H), 7.53 (1H), 8.14 (1H), 8.74 (1H), and 9.13 ppm (1H). MS m/z: 414.1 (M+H).

### EXAMPLES 79-92

### Preparation of Additional β-Oxo-3-pyridinepropanoates

Compounds 49-52, 54, 56-59, 61, 62, 64, 65, and 69 above are prepared in essentially the same manner as described in Example 52 above.

### EXAMPLE 93

### Biological Screening

Fungicidal activity for the compounds described in this invention was determined using a microtiter plate format. In primary screening, test compounds in 1 μL of dimethylsulfoxide (DMSO) are delivered to individual wells of a 96-well microtiter plate. Then 100 μL of minimal media consisting of 1.5% agar is delivered to each well and allowed to cool. Finally, inoculation is carried out by the addition of 10 μL of an aqueous suspension of fungal spores to the surface of the solid agar. The plates are covered and incubated in a controlled environment at 20° C. Fungicidal activity is determined by visual inspection and photometric analysis of fungal growth after 3-5 days, depending on the pathogen. Commercial standards (azoxystrobin, benomyl, captan, chlorothalonil, famoxadone, flusilazole, and propiconazole) are included in all assays. Test pathogens include Septoria tritici, Stagnospora nodorum, Phytophthora infestans, and Botrytis cinerea. Dose response data for compounds found to be fungicidal in primary screening are obtained by screening 3-fold serial dilutions of the test compound. Fungicidal activity, noted as IC50 values in μM concentration, for certain of the compounds covered in this invention is included in the following Table 1. The coefficient of variation (ratio of standard deviation to the mean) expressed in percentage is given in parentheses.

TABLE 1
IC₅₀ Values in μM Concentration and C.V. Values in %

| Compound | | | | | |
|---|---|---|---|---|---|
| Number | B. cinerea | P. infestans | S. nodorum | S. tritici | U. maydis |
| 5 | D | D | A | A (d) | D |
| 15 | NT | D | A (d) | A (d) | D |
| 16 | NT | D | A (b) | A (c) | D |
| 20 | D | D | A (b) | A | D |
| 21 | D | D | A (c) | A (c) | D |
| 29 | D | D (d) | C | A (b) | C |

IC50(μM): A = 0-5; B = 6-15; C = 16-25; D = +25
C.V. (%): (a) = 0-5; (b) = 6-15; (c) = 16-25; (d) = +25
NT = Not Tested

### EXAMPLE 94

### Greenhouse Testing

In planta fungicidal activity for the compounds described in this invention was determined by testing for greenhouse activity against Erysiphe graminis on barley plants and Alternaria solani on tomato plants. For the E. graminis study, barley plants were used at the 2-leaf stage, 7 to 8 inches tall, and 4 plants to a replicate. Commercial control compounds applied were chlorothalonil at 5000 and 1250 ppm and propiconazole at 75, 19, and 5 ppm. Compound 21 was applied at 300 and 75 ppm. All compounds were diluted in 1:1:3 acetone:ethanol:water. Barley plant sets were split into two: one set was inoculated by brushing with a series of E. graminis infested plants, the other was exposed to natural airborne inoculum in a growth chamber. Plants were maintained in a growth chamber under standard conditions for disease development. The trial was rated when the lesions were clearly visible but small enough to be individually counted. The rating consisted of a count of the number of lesions on the 2nd leaf (which was fully expanded at the time of application). Propiconazole gave almost 100% control at the high (75 ppm) rate, with excellent control at the lower rates as well. Chlorothalonil only gave a marginal level of control at the high (5000 ppm) rate, but indicated roughly 50% control at the lower rate, likely due to trace carryover from the high-rate propiconazole treatment which was applied immediately prior. Compound 21 showed excellent activity-against E. graminis at both rates and in both the uninoculated and inoculated tests. Data are presented below in Table 2 for the uninocuated data set and in Table 3 for the inoculated data set:

TABLE 2
Erysiphe Greenhouse Study - uninoculated data set

| | Average Lesion | |
|---|---|---|
| Compound | Number | CI % |
| Chlorothalonil - 5000 ppm | C | c |
| Chlorothalonil - 1250 ppm | B | b |
| Propiconazole - 75 ppm | A | a |
| Propiconazole - 19 ppm | A | a |
| Propiconazole - 5 ppm | A | a |
| Compound 21 - 300 ppm | A | a |
| Compound 21 - 75 ppm | A | a |
| Untreated | D | c |

Mean Lesion Number per 15 cm leaf: A = 0-10; B = 11-20; C = 21-30; D = +30
C.I. (%): a = 0-5; b = 6-15; c = 16-25; d = +25
Ratings taken: 5 days after infection

TABLE 3
Erysiphe Greenhouse Study - inoculated data set

| | Average Lesion | |
|---|---|---|
| Compound | Number | CI % |
| Chlorothalonil - 5000 ppm | D | b |
| Chlorothalonil - 1250 ppm | B | c |
| Propiconazole - 75 ppm | A | a |
| Propiconazole - 18.8 ppm | A | a |
| Propiconazole - 4.7 ppm | A | a |
| Compound 21 - 300 ppm | A | a |
| Compound 21 - 75 ppm | A | b |
| Untreated | D | b |

Mean Lesion Number per 15 cm leaf: A = 0-50; B = 51-100; C = 101-150; D = +150
C.I. (%): a = 0-20; b = 21-40; c = 41-60; d = +60
Ratings taken: 5 days after infection

For the A. solani study, tomato plants were 6 leaf, 8 to 11 inches tall, and 4 plants to a replicate. Commercial control compounds applied were chlorothalonil at 5000 and 1250 ppm and azoxystrobin at 300 and 75 ppm. Compound 21 was applied at 300 and 75 ppm. All compounds were diluted in 1:1:3 acetone: ethanol: water. Tomato plants were inoculated with a conidial suspension (8,000 conidia/ml) of A. solani and maintained in a dew chamber for 24 hours. The plants were subsequently moved to the greenhouse for disease development. The azoxystrobin treatment gave almost 100 percent control at the 300 ppm rate and slightly less control at the 75 ppm rate. The chlorothalonil treatment provided near 100 percent control at both rates (5000 and 1250 ppm). At the low rate (75 ppm), Compound 21 gave no control of A. solani. At the high rate (300 ppm), Compound 21 showed some activity against the disease with a roughly 50% decrease in lesion counts but activity was not significant. The data are presented below in Table 4:

TABLE 4
Alternaria Greenhouse Study

| | Average Lesion | |
|---|---|---|
| Compound | Size | CI % |
| Chlorothalonil - 5000 ppm | A | a |
| Chlorothalonil - 1250 ppm | A | a |
| Azoxystrobin - 300 ppm | A | a |
| Azoxystrobin - 75 ppm | A | b |
| Compound 21 - 300 ppm | C | b |
| Compound 21 - 75 ppm | D | c |
| Untreated | D | b |

Mean Lesion Size: A = 0-10; B = 11-20; C = 21-30; D = +30
C.I. (%): a = 0-5; b = 6-15; c = 16-25; d = +25
Ratings taken: 6 days after infection

The foregoing is illustrative of the present invention, and is not to be construed as limiting thereof. The invention is defined by the following claims, with equivalents of the claims to be included therein.

1. A compound of formula I: 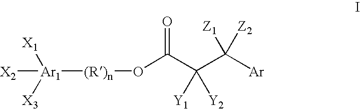
wherein:
Z₁ is —OH and Z₂ is H; or Z₁ and Z₂ together are ═O;
Y₁ and Y₂ are each independently selected from the group consisting of H, alkyl, alkynyl, haloalkyl, alkoxyalkyl, alkylthioalkyl, alkoxy, aryloxy, cyano, R₂R₃NCH₂—, arylthioalkyl, N-heterocycloalkyl, aryl and arylalkyl, each of which can be optionally substituted with halogen, alkyl, alkynyl, haloalkyl, alkoxy, alkylthio, haloalkoxy, cyano, nitro, alkylsulfinyl, or alkylsulfonyl;
or Y₁ and Y₂ taken together form a 3- to 6-membered ring comprised of 3-6 C-atoms, 1-3 N-atoms, and 0-1 O-atom, optionally substituted by alkyl, alkoxycarbonyl, aryl;
Ar is aryl;
Ar₁ is a 5 or 6-membered aromatic or heteroaromatic ring;
X₁, X₂, and X₃ are each taken individually from the group consisting of H, halogen, alkyl, alkenyl, alkynyl, haloalkyl, alkoxy, haloalkoxy, alkylthio, cyano, nitro; aryl or aryloxy or aryloxyalkyl, optionally substituted with halogen, alkyl, haloalkyl, alkoxy, alkylthio, haloalkoxy, cyano, and nitro;
or X₁ and X₂ or X₂ and X₃ as an adjacent pair together form the group —Y—W-Z-, where Y and Z are independently oxygen, sulphur, sulphonyl, carbonyl, or CR₄R₅, W is —(CR₆R₇)ₚ—(CR₈R₉)_{q}— or sulfonyl, R₄ and R₅ are independently hydrogen, halogen, or alkyl, R₆ and R₇ are independently hydrogen, halogen, alkyl or haloalkyl or together form an oxo group, R₈ and R₉ are independently hydrogen, halogen, alkyl or haloalkyl, p is 1 or 2, and q is 0 or 1;
R′ is C1-C4 alkylene optionally substituted with alkyl, haloalkyl, alkoxy, or cyano;
R₂ and R₃ are each taken individually from alkyl, haloalkyl, aryl optionally substituted with halogen, alkyl, alkynyl, haloalkyl, alkoxy, alkylthio, haloalkoxy, cyano, nitro;
or R₂ and R₃ taken together to form a 5 or 6-member heterocycle containing 2-5 C-atoms, 0-1 O-atom, 0-1 S-atom, and 1-3 N-atoms; and
n is 0 or 1;
or a salt thereof.

2. A compound of claim 1 where Z₁ is —OH and Z₂ is H.

3. The compound of claim 1 where Z₁ and Z₂ together are ═O.

4. The compound of claim 1, wherein Y₁ and Y₂ are H or alkyl.

5. The compound of claim 1, wherein at least one of Y₁ and Y₂ are arylalkyl or arylthioalkyl.

6. The compound of claim 1, wherein at least one of Y₁ and Y₂ is aryl.

7. The compound of claim 6, wherein said aryl is substituted aryl.

8. The compound of claim 1, wherein Ar is phenyl, optionally substituted with halogen, alkyl, alkynyl, haloalkyl, alkoxy, alkylthio, haloalkoxy, cyano or nitro.

9. The compound of claim 1, wherein Ar is pyridyl optionally substituted with halogen, alkyl, alkynyl, haloalkyl, alkoxy, alkylthio, haloalkoxy, cyano or nitro.

10. The compound of claim 9, wherein Ar is 3-pyridyl

11. The compound of claim 1, wherein Ar is thiazolyl optionally substituted with halogen, alkyl, alkynyl, haloalkyl, alkoxy, alkylthio, haloalkoxy, cyano or nitro.

12. The compound of claim 1, wherein Ar₁ is selected from the group consisting of phenyl, pyridyl, thienyl, and furyl.

13. The compound of claim 12, wherein Ar₁ is selected from the group consisting of phenyl

14. The compound of claim 1 selected from the group consisting of:

| Compound | | |
|---|---|---|
| Number | Structure | Chemical Name |
| 5 | 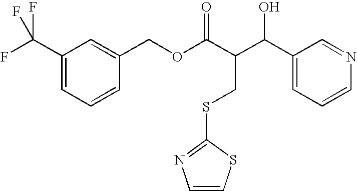 | 3-(Trifluoromethyl)phenylmethyl β-hydroxy-α- (2-thiazolylthiomethyl)-3-pyridinepropanoate |
| 6 | 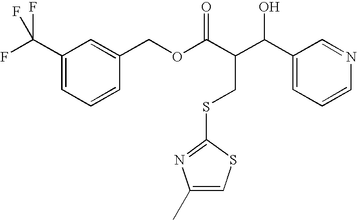 | 3-(Trifluoromethyl)phenylmethyl β-hydroxy-α- (4-methyl-2-thiazolylthiomethyl)-3- pyridinepropanoate |
| 12 | 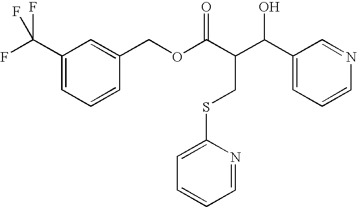 | 3-(Trifluoromethyl)phenylmethyl β-hydroxy-α- (2-pyridylthiomethyl)-3-pyridinepropanoate |
| 15 | 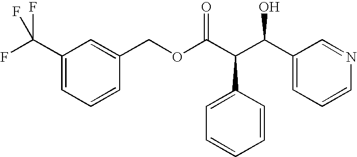 | 3-(Trifluoromethyl)phenylmethyl syn-β-hydroxy-α- phenyl-3-pyridinepropanoate |
| 16 | 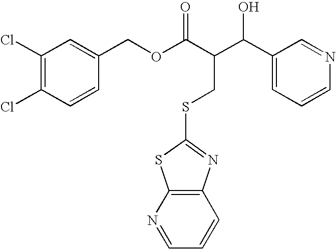 | 3,4-Dichlorophenylmethyl β-hydroxy-α- {2-(thiazolo[5,4-b]pyridine)thiomethyl}-3- pyridinepropanoate |
| 20 | 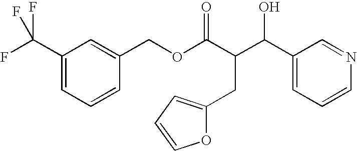 | 3-(Trifluoromethyl)phenylmethyl β-hydroxy-α- 2-furylmethyl-3-pyridinepropanoate |
| 21 | 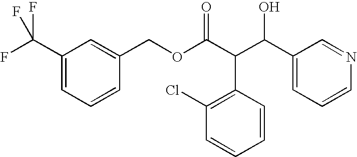 | 3-(Trifluoromethyl)phenylmethyl β-hydroxy-α- 2-chlorophenyl-3-pyridinepropanoate |
| 24 | 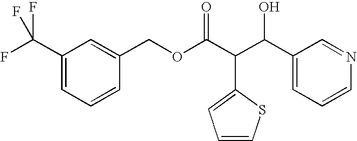 | 3-(Trifluoromethyl)phenylmethyl β-hydroxy-α- (2-thienyl)-3-pyridinepropanoate |
| 29 | 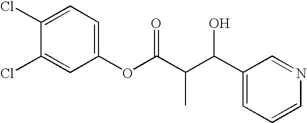 | 3,4-Dichlorophenyl β-hydroxy-α-methyl-3- pyridinepropanoate |
| 44 | 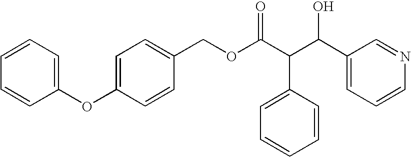 | 4-Phenoxyphenylmethyl β-hydroxy-α- phenyl-3-pyridinepropanoate |
| 46 | 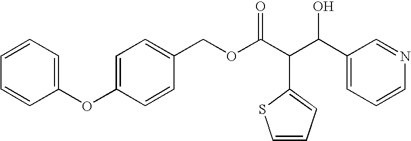 | 4-Phenoxyphenylmethyl β-hydroxy-α- (2-thienyl)-3-pyridinepropanoate |
| 91 | 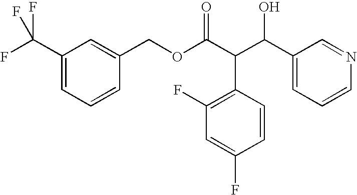 | 3-(Trifluoromethyl)phenylmethyl β-hydroxy- α-(2,4-difluorophenyl)-3-pyridinepropanoate |

and salts thereof.

15. A composition for controlling and preventing plant pathogenic microorganisms comprising, in combination, a compound of claim 1 together with a suitable carrier.

16. The composition of claim 15, further comprising at least one additional fungicide.

17. A method of controlling or preventing infestation of cultivated plants by pathogenic microorganisms, comprising:
applying a compound according to claim 1 to said plants, parts thereof or the locus thereof in an amount effective to control said microorganisms.

18. A method according to claim 17, wherein the microorganism is a fungal organism.

19. The method of claim 17, wherein said fungal organism is selected from the group consisting of Septoria tritici, Stagnospora nodorum, Phytophthora infestans, Ustilago maydis, Botrytis cinerea and Erysiphe graminis.

20. A method of controlling or preventing infestation of plant propagation material by pathogenic microorganisms, comprising:
applying a compound according to claim 1 to said plant propagation material in an amount effective to control said microorganisms.

21. The method of claim 20, wherein said plant propagation material comprises seeds.

22. A method according to claim 20, wherein the microorganism is a fungal organism.

23. The method of claim 22, wherein said fungal organism is selected from the group consisting of Septoria tritici, Stagnospora nodorum, Phytophthora infestans, Ustilago maydis, Botrytis cinerea and Erysiphe graminis.

24. A method of controlling or preventing infestation of a technical material by pathogenic microorganisms, comprising:
applying a compound according to claim 1 to said technical material in an amount effective to control said microorganisms.

25. A method of treating a fungal infection in a subject in need thereof, comprising:
administering a compound of claim 1 or a pharmaceutically acceptable salt thereof to said subject in an amount effective to treat said fungal infection.

26. A method of making a compound of formula Ia: 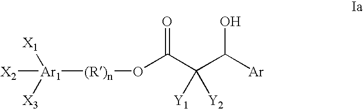
wherein:
Y₁ and Y₂ are each independently selected from the group consisting of H, alkyl, alkynyl, haloalkyl, alkoxyalkyl, alkylthioalkyl, alkoxy, aryloxy, cyano, R₂R₃NCH₂—, arythioalkyl, N-heterocycloalkyl, aryl and arylalkyl, each of which can be optionally substituted with halogen, alkyl, alkynyl, haloalkyl, alkoxy, alkylthio, haloalkoxy, cyano, nitro, alkylsulfinyl, or alkylsulfonyl;
subject to the proviso that at least one of Y₁ and Y₂ is alkyl or arylalkyl;
Ar is aryl;
Ar₁ is a 5 or 6-membered aromatic or heteroaromatic ring;
X₁, X₂, and X₃ are each taken individually from the group consisting of H, halogen, alkyl, alkenyl, alkynyl, haloalkyl, alkoxy, haloalkoxy, alkylthio, cyano, nitro; aryl or aryloxy or aryloxyalkyl, optionally substituted with halogen, alkyl, haloalkyl, alkoxy, alkylthio, haloalkoxy, cyano, and nitro;
or X₁ and X₂ or X₂ and X₃ as an adjacent pair together form the group —Y—W-Z-, where Y and Z are independently oxygen, sulphur, sulphonyl, carbonyl, or CR₄R₅, W is —(CR₆R₇)ₚ—(CR₈R₉)_{q}— or sulfonyl, R₄ and R₅ are independently hydrogen, halogen, or alkyl, R₆ and R₇ are independently hydrogen, halogen, alkyl or haloalkyl or together form an oxo group, R₈ and R₉ are independently hydrogen, halogen, alkyl or haloalkyl, p is 1 or 2, and q is 0 or 1;
R′ is C1-C4 alkylene optionally substituted with alkyl, haloalkyl, alkoxy, or cyano;
R₂ and R₃ are each taken individually from alkyl, haloalkyl, aryl optionally substituted with halogen, alkyl, alkynyl, haloalkyl, alkoxy, alkylthio, haloalkoxy, cyano, nitro;
or R₂ and R₃ taken together to form a 5 or 6-member heterocycle containing 2-5 C-atoms, 0-1 O-atom, 0-1 S-atom, and 1-3 N-atoms; and
n is 0 or 1;
comprising:
condensing an ester of formula III: 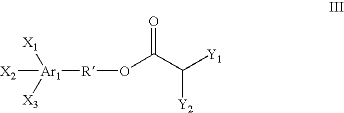
with an aldehyde of formula IV: 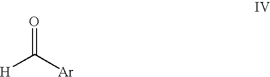
to produce said compound of formula Ia.

27. A method of making a compound of formula anti-Ia: 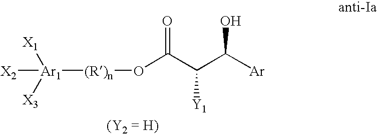
wherein:
Y₁ is alkyl, aryl, arylalkyl, alkoxy, aryloxy, or cyano;
Ar is aryl;
Ar₁ is a 5 or 6-membered aromatic or heteroaromatic ring;
X₁, X₂, and X₃ are each taken individually from the group consisting of H, halogen, alkyl, alkenyl, alkynyl, haloalkyl, alkoxy, haloalkoxy, alkylthio, cyano, nitro; aryl or aryloxy or aryloxyalkyl, optionally substituted with halogen, alkyl, haloalkyl, alkoxy, alkylthio, haloalkoxy, cyano, and nitro;
or X₁ and X₂ or X₂ and X₃ as an adjacent pair together form the group —Y—W-Z-, where Y and Z are independently oxygen, sulphur, sulphonyl, carbonyl, or CR₄R₅, W is —(CR₆R₇)ₚ—(CR₈R₉)_{q}— or sulfonyl, R₄ and R₅ are independently hydrogen, halogen, or alkyl, R₆ and R₇ are independently hydrogen, halogen, alkyl or haloalkyl or together form an oxo group, R₈ and R₉ are independently hydrogen, halogen, alkyl or haloalkyl, p is 1 or 2, and q is 0 or 1;
R′ is C1-C4 alkylene optionally substituted with alkyl, haloalkyl, alkoxy, or cyano; and
n is 0 or 1;
comprising:
condensing an ester of formula III: 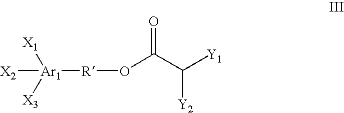
wherein Y₁ is H
with an aldehyde of formula IV: 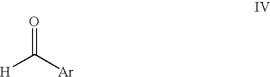
in a boron-mediated aldol reaction to produce said compound of formula anti-Ia.

28. A method of making a compound of formula syn-Ia: 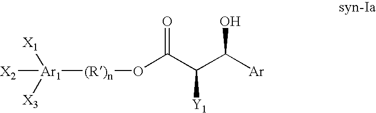
wherein:
Y₁ is alkyl, aryl, arylalkyl, alkoxy, aryloxy, or cyano;
Ar is aryl;
Ar₁ is a 5 or 6-membered aromatic or heteroaromatic ring;
X₁, X₂, and X₃ are each taken individually from the group consisting of H, halogen, alkyl, alkenyl, alkynyl, haloalkyl, alkoxy, haloalkoxy, alkylthio, cyano, nitro; aryl or aryloxy or aryloxyalkyl, optionally substituted with halogen, alkyl, haloalkyl, alkoxy, alkylthio, haloalkoxy, cyano, and nitro;
or X₁ and X₂ or X₂ and X₃ as an adjacent pair together form the group —Y—W-Z-, where Y and Z are independently oxygen, sulphur, sulphonyl, carbonyl, or CR₄R₅, W is —(CR₆R₇)ₚ—(CR₈R₉)_{q}— or sulfonyl, R₄ and R₅ are independently hydrogen, halogen, or alkyl, R₆ and R₇ are independently hydrogen, halogen, alkyl or haloalkyl or together form an oxo group, R₈ and R₉ are independently hydrogen, halogen, alkyl or haloalkyl, p is 1 or 2, and q is 0 or 1;
R′ is C1-C4 alkylene optionally substituted with alkyl, haloalkyl, alkoxy, or cyano;
n is 0 or 1;
comprising:
condensing an ester of formula III: 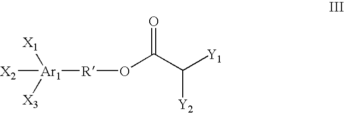
wherein Y₂ is H
with an aldehyde of formula IV: 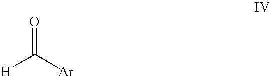
in a boron-mediated aldol reaction to produce said compound of formula syn-Ia.

29. A method of making a compound of formula Ia: 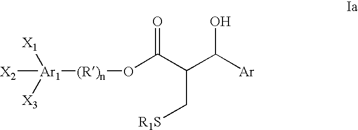
wherein:
R₁ is alkyl or aryl;
Ar is aryl;
Ar₁ is a 5 or 6-membered aromatic or heteroaromatic ring;
X₁, X₂, and X₃ are each taken individually from the group consisting of H, halogen, alkyl, alkenyl, alkynyl, haloalkyl, alkoxy, haloalkoxy, alkylthio, cyano, nitro; aryl or aryloxy or aryloxyalkyl, optionally substituted with halogen, alkyl, haloalkyl, alkoxy, alkylthio, haloalkoxy, cyano, and nitro;
or X₁ and X₂ or X₂ and X₃ as an adjacent pair together form the group —Y—W-Z-, where Y and Z are independently oxygen, sulphur, sulphonyl, carbonyl, or CR₄R₅, W is —(CR₆R₇)ₚ—(CR₈R₉)_{q}— or sulfonyl, R₄ and R₅ are independently hydrogen, halogen, or alkyl, R₆ and R₇ are independently hydrogen, halogen, alkyl or haloalkyl or together form an oxo group, R₈ and R₉ are independently hydrogen, halogen, alkyl or haloalkyl, p is 1 or 2, and q is 0 or 1;
R′ is C1-C4 alkylene optionally substituted with alkyl, haloalkyl, alkoxy, or cyano;
n is 0 or 1;
comprising:
reacting a substituted thiol of the formula R₁SH with an acrylate of formula V: 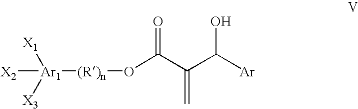
in a Michael addition to produce said compound of formula Ia.

30. A method of making a compound of formula Ia: 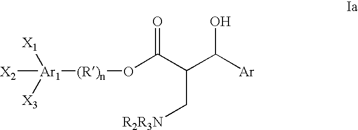
wherein:
R₂ and R₃ are each taken individually from alkyl; haloalkyl; and aryl optionally substituted with halogen, alkyl, alkynyl, haloalkyl, alkoxy, alkylthio, haloalkoxy, cyano, nitro; or R₂ and R₃ taken together form a 5 or 6-member heterocycle containing 2-5 C-atoms, 0-1 O-atom, 0-1 S-atom, and 1-3 N-atoms;
Ar is aryl;
Ar₁ is a 5 or 6-membered aromatic or heteroaromatic ring;
X₁, X₂, and X₃ are each taken individually from the group consisting of H, halogen, alkyl, alkenyl, alkynyl, haloalkyl, alkoxy, haloalkoxy, alkylthio, cyano, nitro; aryl or aryloxy or aryloxyalkyl, optionally substituted with halogen, alkyl, haloalkyl, alkoxy, alkylthio, haloalkoxy, cyano, and nitro;
or X₁ and X₂ or X₂ and X₃ as an adjacent pair together form the group —Y—W-Z-, where Y and Z are independently oxygen, sulphur, sulphonyl, carbonyl, or CR₄R₅, W is —(CR₆R₇)ₚ—(CR₈R₉)_{q}— or sulfonyl, R₄ and R₅ are independently hydrogen, halogen, or alkyl, R₆ and R₇ are independently hydrogen, halogen, alkyl or haloalkyl or together form an oxo group, R₈ and R₉ are independently hydrogen, halogen, alkyl or haloalkyl, p is 1 or 2, and q is 0 or 1;
R′ is C1-C4 alkylene optionally substituted with alkyl, haloalkyl, alkoxy, or cyano; and
n is 0 or 1;
comprising:
reacting a substituted amine of the formula R₂R₃NH with an acrylate of formula V: 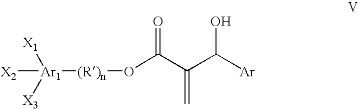
in a Michael addition to produce said compound of formula Ia.

31. A method of making an acrylate of formula V: 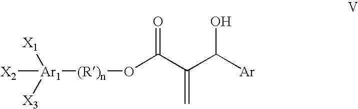
wherein:
Ar is aryl;
Ar₁ is a 5 or 6-membered aromatic or heteroaromatic ring;
X₁, X₂, and X₃ are each taken individually from the group consisting of H, halogen, alkyl, alkenyl, alkynyl, haloalkyl, alkoxy, haloalkoxy, alkylthio, cyano, nitro; aryl or aryloxy or aryloxyalkyl, optionally substituted with halogen, alkyl, haloalkyl, alkoxy, alkylthio, haloalkoxy, cyano, and nitro;
or X₁ and X₂ or X₂ and X₃ as an adjacent pair together form the group —Y—W-Z-, where Y and Z are independently oxygen, sulphur, sulphonyl, carbonyl, or CR₄R₅, W is —(CR₆R₇)ₚ—(CR₈R₉)_{q}— or sulfonyl, R₄ and R₅ are independently hydrogen, halogen, or alkyl, R₆ and R₇ are independently hydrogen, halogen, alkyl or haloalkyl or together form an oxo group, R₈ and R₉ are independently hydrogen, halogen, alkyl or haloalkyl, p is 1 or 2, and q is 0 or 1;
R′ is C1-C4 alkylene optionally substituted with alkyl, haloalkyl, alkoxy, or cyano;
n is 0 or 1;
comprising:
reacting an unsubstituted acrylate ester of formula VIII: 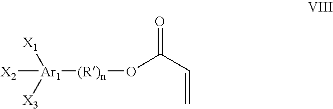
with an aldehyde of formula IV 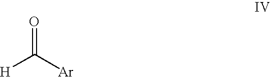
in a Baylis-Hillman reaction to produce said acrylate of formula V.

32. A method of making a compound of formula II: 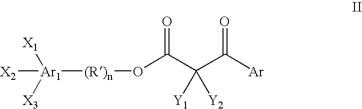
wherein:
Y₁ and Y₂ are each independently selected from the group consisting of H, alkyl, alkynyl, haloalkyl, alkoxyalkyl, alkylthioalkyl, alkoxy, aryloxy, cyano, R₂R₃NCH₂—, arythioalkyl, N-heterocycloalkyl, aryl and arylalkyl, each of which can be optionally substituted with halogen, alkyl, alkynyl, haloalkyl, alkoxy, alkylthio, haloalkoxy, cyano, nitro, alkylsulfinyl, or alkylsulfonyl;
or Y₁ and Y₂ taken together form a 3- to 6-membered ring comprised of 3-6 C-atoms, 1-3 N-atoms, and 0-1 O-atom, optionally substituted by alky, alkoxycarbonyl, aryl;
Ar is aryl;
Ar₁ is a 5 or 6-membered aromatic or heteroaromatic ring;
X₁, X₂, and X₃ are each taken individually from the group consisting of H, halogen, alkyl, alkenyl, alkynyl, haloalkyl, alkoxy, haloalkoxy, alkylthio, cyano, nitro; aryl or aryloxy or aryloxyalkyl, optionally substituted with halogen, alkyl, haloalkyl, alkoxy, alkylthio, haloalkoxy, cyano, and nitro;
or X₁ and X₂ or X₂ and X₃ as an adjacent pair together form the group —Y—W-Z-, where Y and Z are independently oxygen, sulphur, sulphonyl, carbonyl, or CR₄R₅, W is —(CR₆R₇)ₚ—(CR₈R₉)_{q}— or sulfonyl, R₄ and R₅ are independently hydrogen, halogen, or alkyl, R₆ and R₇ are independently hydrogen, halogen, alkyl or haloalkyl or together form an oxo group, R₈ and R₉ are independently hydrogen, halogen, alkyl or haloalkyl, p is 1 or 2, and q is 0 or 1;
R′ is C1-C4 alkylene optionally substituted with alkyl, haloalkyl, alkoxy, or cyano;
R₂ and R₃ are each taken individually from alkyl, haloalkyl, aryl optionally substituted with halogen, alkyl, alkynyl, haloalkyl, alkoxy, alkylthio, haloalkoxy, cyano, nitro;
or R₂ and R₃ taken together to form a 5 or 6-member heterocycle containing 2-5 C-atoms, 0-1 O-atom, 0-1 S-atom, and 1-3 N-atoms; and
n is 0 or 1;
comprising oxidizing a compound of formula Ia: 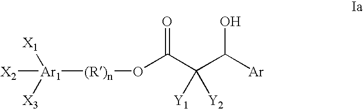
to produce said compound of formula II.
